(19) United States
(12) Patent Application Publication
(10) Pub. No.: US 2025/0382414 A1
(43) Pub. Date: Dec. 18, 2025

(54) RECYCLABLE AND MALLEABLE THERMOSETS ENABLED BY ACTIVATING DORMANT DYNAMIC LINKAGES

(71) Applicant: THE REGENTS OF THE UNIVERSITY OF COLORADO, A BODY CORPORATE, Denver, CO (US)

(72) Inventors: Wei ZHANG, Boulder, CO (US); Zepeng LEI, Boulder, CO (US)

(21) Appl. No.: 18/877,173

(22) PCT Filed: Jun. 21, 2023
(86) PCT No.: PCT/US2023/025899

§ 371 Date: Dec. 19, 2024

Related U.S. Application Data
(60) Provisional application No. 63/354,754, filed on Jun. 23, 2022.

(51) Int. Cl. C08G 65/42; C07D 251/30; C07D 487/04; C07D 487/22

(52) U.S. Cl. CPC C08G 65/42; C07D 251/30; C07D 487/04; C07D 487/22

(57) ABSTRACT

The invention disclosed herein relates a novel class of alkyl- and/or aryl-linked crosslinked polymeric polycyanurate compounds and their methods of synthesis from alkoxy substituted triazines by reacting said alkoxy substituted triazines with diols. Further provided a method of synthesis of an alkyl-linked polyarylether monomer/network comprising reacting the alkoxy substituted phenyl derivatives having electron withdrawing group, with diols.

### CROSS REFERENCE TO RELATED APPLICATIONS

This International PCT application claims the benefit of and priority to U.S. Provisional Application No. 63/354,754, filed Jun. 23, 2022. The specification, claims and drawings of which are incorporated herein by reference in their entirety.

### TECHNICAL FIELD

Generally, the inventive technology disclosed herein relates novel alkyl- and/or aryl-linked polycyanurate compounds and their methods of synthesis.

### BACKGROUND OF THE INVENTION

Plastics have become an indispensable part of our daily life. The properties such as lightweight, durability, excellent barrier properties, and low cost in most plastics have brought tremendous social benefits and technological advances. However, the ever-increasing demand for plastics, the negative environmental impact of their uncontrolled disposal, and the challenges in recycling have raised alarming concerns over their detrimental long-term effects on the environment and human health. Realizing recyclability of polymeric materials to achieve circular economy and environmental sustainability has attracted great attention. Thermosets with monomers permanently crosslinked by strong covalent bonds are usually produced as by-design non-processable and nonrecyclable plastics. To solve their recyclability problem, there has been an explosion of creating novel thermosetting polymers via introducing cleavable or dynamic covalent bonds into monomers or as crosslinkers. Various chemistries such as dynamic imine bonds, transesterification, boronic ester bonds, urethane, and silyl ether bonds have been explored as cleavable units to prepare those materials. However, research that focuses on achieving recyclability and malleability of existing thermosets to realize their circular economy remains scarce. Although it is important to develop new polymers as “green” replacements, revisiting traditional materials with new aspects brought in is crucial and can generate critical knowledge guiding the development of sustainable high-performance materials.

Retrosynthetic analysis has long been the norm for synthetic chemists to evaluate the possible disconnection choices of target structures and find the most efficient route. However, the importance of retrosynthetic analysis in polymer synthesis is often overlooked because they are made of simple repeating units with limited possible connectivity. By searching backward, alternative synthetic pathways can be found for polymers, which could offer unexpected benefits. For example, poly(phenyleneethynylene)s (PPEs) were typically synthesized through cross-coupling reactions, which generally provide PPEs with relatively low molecular weight and diyne defects (FIG. 1a). However, by forming C≡C bonds instead of C—C bonds through alkyne metathesis, defect-free PPEs with high molecular weight can be obtained. More importantly, by employing dynamic alkyne metathesis, depolymerization of PPEs into small molecules and their potential closed-loop recyclability could be possible. Recent studies on chemically recyclable polymers and covalent adaptable networks (CANs) show that the activation of reversibility for bond connections in polymers could be the key driver of recyclability and malleability of thermosetting polymers. Therefore, the present inventors sought to uncover potentially reversible bonds through retrosynthetic analysis in traditional thermosetting polymers.

As a proof of concept, the present invention selected cyanate ester resins as an exemplary model system to demonstrate our strategy of achieving recyclability and malleability by activating dormant dynamic linkages for traditional thermosets. Polycyanurate networks (PCNs) have been widely employed in aerospace and microelectronics industries, and their market is expected to reach 338 million dollars in 2022. Cyanate ester resins are traditionally cured through [2+2+2]cyclotrimerization of three cyanate groups (FIG. 1b) to form polycyanurate networks (PCNs), which exhibit unique properties such as good flame retardancy, high thermal stability, low moisture absorption, low dielectric constant dissipation factors, excellent compatibility with carbon fibers, and adherence to metals. Recycling such crosslinked thermosets is challenging. Previously, PCNs were degraded into triazine-based structures and phenols by treatment with various nucleophiles. The resulting products are obtained as mixtures, which can be further used in polyurethane synthesis. However, closed-loop recycling of PCNs back to clean and reusable building blocks (e.g., triazine-based monomers for repolymerization) has never been achieved. In addition, because alkyl-O—C≡N monomers undergo isomerization under the high temperature, [2+2+2]cyclotrimerization is an irreversible reaction with the substrate scope limited to aromatic aryl-O—C≡N monomers, generally providing highly brittle PCNs. Alkyl groups could only be uncontrollably introduced into PCNs via the conventional trimerization method, and alkyl-linked PCNs therefore still remain untapped due to the difficulties in their synthesis.

By performing retrosynthetic analysis and rethinking the possible alternative routes, the present inventors envision that PCNs can also be constructed by forming the single bond between triazine carbon and oxygen through nucleophilic aromatic substitution (S_{N}Ar). Here, we demonstrate that by adopting reversible S_{N}Ar chemistry instead of irreversible cyclotrimerization, the dormant dynamic linkage in PCNs can be activated. Therefore, recyclable and malleable PCNs can be prepared from two simple building blocks, and upcycling traditional aryl PCNs to reusable monomers for alkyl PCN synthesis is possible. Through this new synthetic route, PCN synthesis is not limited to only aryl linkers, thus offering unprecedented tunability in PCN properties, which has proven technically challenging when traditional cyclotrimerization approach is used. The alkyl PCNs show excellent film properties, chemical resistance, and recyclability. End-of-life PCNs in the mixed plastic waste stream could be selectively degraded back to the starting monomers, which can be separated and directly reused in the next production cycle, achieving closed-loop polymer-to-polymer recycling.

### SUMMARY OF INVENTION

In one aspect, the present invention includes novel thermoset polymer compositions. In a preferred embodiment, the thermoset polymers of the invention one or more novel alkyl- and/or aryl-linked polycyanurate networks (PCNs) compositions. In one aspect, alkyl-linked PCN monomer compounds of the invention may synthesize through a reversible S_{N}Ar reaction between alcohols and cyanurates. In another aspect, the alkyl-linked PCN s can be converted to monomers through refluxing in alcohol, and preferably ethanol. In this aspect, potassium carbonate may be use as a base to deprotonate the ethanol and accelerate the conversion.

In a preferred aspect, the thermoset polymers of the invention contain one or more novel polyarylether (PAE) compositions. In this aspect, PAE monomers compounds of the invention can be synthesized through a reversible S_{N}Ar reaction between alcohols and di/triarylether with two or more cyano, aldehyde, and/or halogen groups. In another aspect, the PAEs can be converted to monomers through refluxing in alcohol, and preferably methanol. In this aspect, potassium carbonate may be use as a base to deprotonate the ethanol and accelerate the conversion.

In another aspect, the present invention includes novel systems, methods for the synthesis of novel thermoset polymer compositions. In a preferred embodiment, the invention includes the use of reversible nucleophilic aromatic substitution (S_{N}Ar) to synthesize novel alkyl- and/or aryl-linked PCNs.

In a preferred embodiment, the invention includes the synthesis of an aryl-linked PCNs formed by the replacement of an ethoxy group on a cyanurate structure with bisphenol A (BPA).

In a preferred embodiment, the invention includes the synthesis of an alkyl-linked PCNs formed by the replacement of an ethoxy group on a cyanurate structure with an alkyl diol. In a preferred aspect, the alkyl diol may be selected from 1,4-butandiol (DO-4), 1,6-hexandiol (DO-6) and 1,12-dodecanediol (DO-12) which may act as linkers through S_{N}Ar reaction as described herein.

Additional aspects of the invention include the methods of upcycling traditional aryl PCNs to reusable monomers for alkyl PCN synthesis. In a preferred embodiment, a used aryl-PCN material may be converted to a cyanurate structure with the ethoxy group replaced by one or more alkyl diols forming a novel alkyl (PCNs).

Additional embodiments of the invention include methods of converting an alkyl-lined PCNs into its monomer subunits, preferably through refluxing the PCNs in ethanol in the presence of a potassium carbonate catalyst.

Additional aspects of the invention may become evident based on the specification and figures presented below.

### BRIEF DESCRIPTION OF THE DRAWINGS

FIG. 1A-B. Synthetic strategies of polymers. a, Poly(phenyleneethynylene)s (PPEs) can be prepared either through cross-coupling between aryl halide and terminal alkynes (blue) or alkyne metathesis polymerization (red). b, Polycyanurate networks (PCNs) can be prepared through [2+2+2]-cyclotrimerization of cyanate esters (blue) or dynamic S_{N}Ar reaction between alkoxyl triazine and alcohol (red). [2+2+2]-cyclotrimerization is an irreversible reaction and the method is limited to the synthesis of aryl PCNs. By contrast, the reversibility in S_{N}Ar reaction can be activated under certain conditions, thus enabling malleability and recyclability of polycyanurate networks. Both aryl PCNs and alkyl PCNs are accessible through S_{N}Ar reaction.

FIG. 2A-C. S_{N}Ar in cyanurate exchange reactions. a, When 6 mol % of TBD was added, the exchange reaction TETA and methanol occurred and reached equilibrium within 40 hours at 60° C. Three new types of triazines, which are substituted with one, two, or three methoxy groups, were formed. b, Kinetic profiles of the S_{N}Ar reaction at different temperatures were obtained by plotting the relative concentration (% of remained TETA) of TETA vs. time. c, Arrhenius plot and its linear fitting of the small molecule analog reaction. The activation energy is determined to be 62.5 kJ/mol.

FIG. 3A-F. Preparation and characterizations of PCNs. a, TETA monomer can be either obtained from depolymerization of used Aryl-PCN prepared via conventional trimerization or synthesized from commercially available cyanuric chloride. The alkyl-PCNs were synthesized through S_{N}Ar reaction between TETA and various diols in anisole. b, Representative stress-strain curves of the PCNs. c, Tan δ-temperature curves obtained through DMA tests of the PCNs. d, Gel fraction test of the PCNs. e, Comparison of FT-IR comparison spectra of PCN-A6 after different solution treatments for 48 hours. f, Transparent film of PCN-A6 can be used as a chemical-resistant film. After spills of different solvents (acetone, dichloromethane, and ethanol), PCN-A6 retained the same transparency while polystyrene is severely damaged.

FIG. 4A-E. Chemical recycling of PCNs. a, Closed-loop recycling of PCNs. The PCNs can be depolymerized into monomers, which can be repolymerized to form recycled PCNs with nearly identical chemical, thermal, and mechanical properties. Reversible S_{N}Ar reaction thus enables a closed-loop polymer-polymer recycling of PCNs. b, Photographs showing selective recycling procedures of PCN-A6 from the plastic waste containing HDPE, PP, and PS. PCN-A6 was cleanly converted to soluble monomers, while other plastics remained as solids. The recycled TETA was reused to form PCN-A6. c, Comparison of the ¹H-NMR spectra of recycled and freshly made TETA. Recycled TETA shows identical NMR proton signals as the freshly synthesized TETA. d, Mechanical properties of the virgin PCNs and recycled PCNs are highly comparable; e, Recycled PCNs exhibit nearly identical glass transition temperatures as the virgin PCNs.

FIG. 5. Closed-loop recycling of PCNs via dynamic S_{N}Ar. PCNs can be made through polymerization of diols and substituted triazine monomers. When treated with mono alcohol or mono phenol, the PCNs can be converted to the monomer mixture. If needed, the monomer mixture can be further separated and purified. Traditional irreversible trimerization method is only applicable to aryl PCNs because alkyl-OCNs undergo fast isomerization under the reaction conditions.

FIG. 6A-B. Small molecule study of cyanurate exchange. a, No reaction between TETA and methanol was observed without TBD catalyst. b, The first step of exchange reaction between TETA and deuterated methanol can be considered as an irreversible pseudo first-order reaction when the deuterated methanol is used as solvent.

FIG. 7A-C. FTTR comparison. a, FTTR spectra of PCN-A4 film and its corresponding monomers. b, FTIR spectra of PCN-A6 film and its corresponding monomers. c, FTIR spectra of PCN-A12 film and its corresponding monomers.

FIG. 8A-C. Chemical resistance test of PCN films. a, Chemical resistance test for PCN-A4 film. b, Chemical resistance test for PCN-A6 film. c, Chemical resistance test for PCN-A12 film. The PCN films were cut into the rectangular shape and submerged in different solutions (1M HCl, 1M NaOH, 30% H₂O₂ and 1M NaBH4); the top, middle and bottom photos were taken before submerging, after 48-hour submerging, and after drying, respectively. No change in appearance was observed for all the PCN films.

FIG. 9A-C. Chemical recycling of PCN-A12. a, ¹H-NMR spectra show that the film degradation in ethanol is clean (TMB, 1,3,5-trimethoxybenzene, used as internal standard) and the recycled DO-12 and TETA are in high purity. b, Nearly identical loss factors for the original and recycled PCN-A12 samples. c, Nearly identical FTIR spectra of the original and recycled PCN-A12 samples.

FIG. 10A-C. Chemical recycling of PCN-A4. a, 1H-NMR spectra show that the film degradation in ethanol is clean (TMB used as internal standard). and the recycled TETA is in high purity. b, Nearly identical loss factors for the original and recycled PCN-A4 samples. c, Nearly identical FTIR spectra of the original and recycled PCN-A4 samples.

FIG. 11A-C. Chemical recycling of PCN-A6. a, ¹H-NMR spectra show that the film degradation in ethanol is clean (TMB used as internal standard) and the recycled TETA is in high purity. b, Neary identical loss factor for the original and recycled PCN-A6 samples. c, Nearly identical FTIR spectra of the original and recycled PCN-A6 samples.

FIG. 12A-C. Kinetic study of cyanurate exchange in solid state. a, Bond exchange reaction can be triggered under heat in the presence of 23 mol % excess of diol monomers and 10 mol % of TBD. b, Stress relaxation test of PCN-A6-m at various temperatures. c, Arrhenius plot and its linear fitting. The activation energy was calculated to be 76.7 kJ/mol.

FIG. 13. ¹H-NMR spectrum of TETA.

FIG. 14. ¹³C-NMR spectrum of TETA.

FIG. 15. ¹H-NMR spectrum of the TPhTA prepared via phenol exchange.

FIG. 16. ¹H-NMR spectrum of the residue.

FIG. 17. ¹H-NMR spectrum of the mixture after stirring at 100° C. for 16 hours.

FIG. 18. FT-IR spectra of DCBPA and PCN-DCBPA.

FIG. 19. ¹H-NMR spectra of (a) crude reaction mixture from PCN-DCPBA depolymerization, (b) TETA recovered from PCN-DCBPA upcycling and (c) Bisphenol A recovered from PCN-DCBPA upcycling.

FIG. 20A-E. ¹H-NMR spectra of the mixtures in the kinetic studies at (a) 20° C., (b) 35° C., (c) 40° C., (d) 45° C., and (e) 50° C.

FIG. 21A-B. ln([C]₀/[C]) vs. t plots of cyanurate exchange at (a) 35° C., 40° C., 45° C., and 50° C. and (b) 20° C.

FIG. 22A-B. (a) Reaction of TETA with three equivalent methanol. (b) GC-MS spectra of the pure TETA (top) and the reaction mixture (bottom) from (a).

FIG. 23A-B. (a) Reaction of TETA in deuterated methanol (as solvent). (b) GC-MS spectra of the pure TETA (top) and the reaction mixture (bottom) from (a).

FIG. 24. CP/MAS NMR spectra of PCNs (Teflon signal from NMR vessel was observed at 116 ppm). The unreacted —OEt group percentages was estimated from the relative peak intensities to be less than 10 mol % for PCN-A4, PCN-A6 and PCN-A12, which is consistent with the mass calculations above.

FIG. 25. Tensile test curves of PCNs FIG. 26A-C. Storage modulus and Tan delta curves of PCNs. (a) PCN-A4; (b) PCN-A6; (c) PCN-A12.

FIG. 27A-C. DSC results indicate the T_{g}s (onset) are 63° C., 44° C. and −3° C. for PCN-A4, PCN-A6 and PCN-A12, respectively.

FIG. 28A-B. FTIR spectra of PCNs before (light grey) and after treatment (bottom): (a) PCN-A4; (b) PCN-A12.

FIG. 29A-B. UV-Vis spectra of PCN-A6, PS and PSU. (a) Transmittance measurement before solvent spill. (b) Transmittance measurement after solvent spills.

FIG. 30. TGA of PCNs shows <4 wt % of mass loss below 300° C.

FIG. 31A-B. Characterizations of PCN-BPA. (a) Gel fraction test; (b) FT-IR spectra.

FIG. 32. ¹H-NMR spectrum of the degraded PCN-A4 mixture. TETA and DO-4 are approximately in 2:3 molar ratio.

FIG. 33. ¹H-NMR spectrum of the degraded PCN-A6 mixture. TETA and DO-6 are approximately in 2:3 molar ratio.

FIG. 34. ¹H-NMR spectrum of the degraded PCN-A12 mixture. TETA and DO-12 are approximately in 2:3 molar ratio.

FIG. 35. ¹H-NMR spectrum of recycled TETA from PCN-A6 with plastic waste stream.

FIG. 36. Tensile test curves of recycled PCNs.

FIG. 37A-C. Storage modulus and Tan delta curves of recycled PCNs. (a) PCN-A4; (b) PCN-A6; (c) PCN-A12.

FIG. 38A-D. Reprocessing PCN-A6-m. (a) Images of forming PCN-A6-m strip from small polymer pieces. (b) FTIR spectra of original PCN-A6-m, reprocessed PCN-A6-m and PCN-A6 (c) Tensile test curves of PCN-A6-m. (d). Storage modulus and Tan delta curves of PCN-A6-m (solid: original; dash: reprocessed).

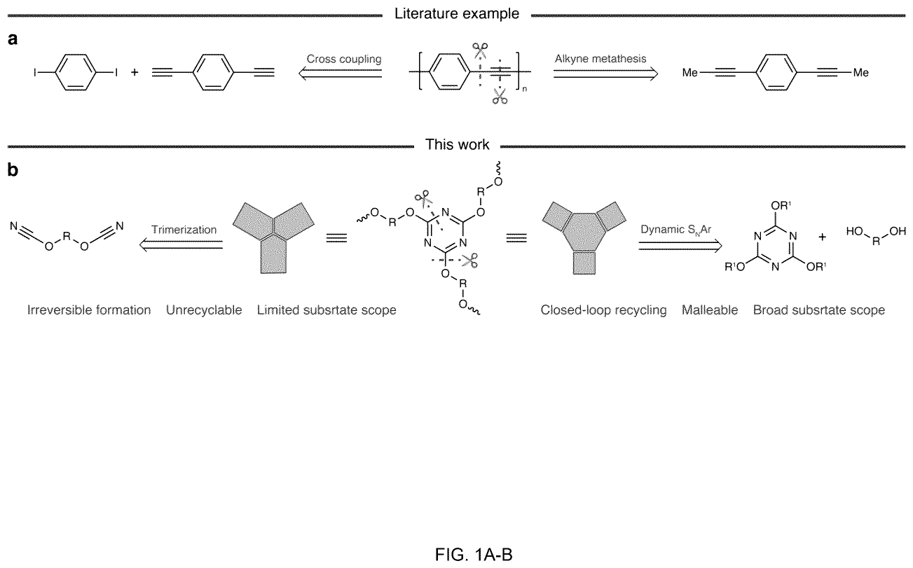

### DETAILED DESCRIPTION OF THE INVENTION

As noted above, polymer re-use has been deemed highly critical for improving plastics circular economy and environmental sustainability. Chemical recycling has attracted increasing interests due to its capability of degrading polymers to precursors and building blocks, which could be used as feedstocks similar to petroleum-based chemicals. Although there are several approaches towards creating novel recyclable polymers via introducing cleavable or dynamic linkers, existing thermoset polymers have been widely overlooked since they are considered as permanently bonded materials. Herein, by performing the retrosynthetic analysis of a traditional polycyanurate thermoset, the present inventors redirected the synthetic route from conventional C—N bond formation via irreversible cyanate trimerization to constructing the C—O bonds through reversible nucleophilic aromatic substitution between alkoxyl triazine and alcohol. This approach for polycyanurate synthesis overcomes a number of limitations in traditional trimerization approach, including the substrate scope limited only to aryl monomers, high reaction temperature, and the difficulty of remolding, repairing, and recycling. Previously inaccessible alkyl-polycyanurate thermosets have been successfully prepared, which show excellent film properties with high chemical resistance under various conditions, and closed-loop polymer-to-polymer recyclability. The results described in this invention reveal that revisiting the chemical structures of traditional thermosetting polymers, assisted with retrosynthetic analysis, could lead to discoveries of “apparently dormant” dynamic linkages. Utilizing those dynamic linkages to construct the same type of polymer network would significantly expand the monomer scope and enable sustainable features for traditionally non-recyclable materials, without sacrificing their physical properties.

In one embodiment, the invention includes an alkyl- and/or aryl-linked crosslinked polymeric compound according to Formula (I) comprising:

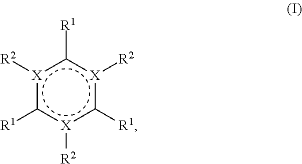

wherein
X is independently N, or C, and further when X are all N, then R² is absent, and when X is all C then R² is present;
R¹ is independently CH, halogen, alkyl, or diol selected from alkyl diol or aryl diol, and wherein at least two of R¹ are independently alkyl diol, or aryl diol;
R² is independently H, CH, or an electron withdrawing group, and wherein at least two of R² are independently an electron withdrawing group;
wherein any of R¹ and R² optionally form together one or more an aromatic rings, or one or more heterocyclic rings, and wherein the one or more rings are optionally substituted with at least one electron withdrawing group, and wherein the one or more rings of the compound further form an electron deficient ringed core; and
wherein said dashed lines represent possible double bond positions according to the configuration of X being N or C, wherein the double bond positions form an aromatic ring.

In another embodiment, the compound according to Formula (I) can include a compound wherein R¹ is selected from: polyol, polythiol, bisphenol A, polyamine, 1,4-butandiol, 1,6-hexandiol, and 1,12-dodecanediol, or a combination of the same. In another embodiment, R¹ of the compound of Formula (I) can be selected from:

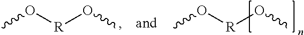

wherein R is C₄₋₁₂ linear alkyl, an aromatic diol, polyol, polythiol or polyamine; and
n is greater than one.

In another embodiment, the compound according to Formula (I) can include an electron withdrawing group selected from: NO₂, CN, CHO, halogen, CO₂R³, CONR³, CH═NR³, (C═S)OR³, (C═O)SR³, CS₂R³, SO₂R³, SO₂NR³, SO₃R³, P(O)(OR³)₂, P(O)(R³)₂, or B(OR³)₃, wherein R³ is an alkyl, an aryl or H. In a preferred embodiment, the compound according to Formula (I) can include an electron withdrawing group selected from: CN, CHO, or halogen.

As shown above, in a preferred embodiment, the core of the compound of Formula (I) is an aromatic ring, which can include additional ring structures formed between R¹ and R² as described above. Notably, in this embodiment, the core aromatic ring is electron deficient. As an example, the compound according to Formula I can include the following exemplary compounds having electron deficient core aromatic ring structures:

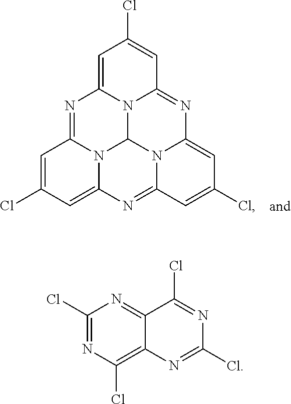 [SMILES: ClC1=CC2=NC3=CC(Cl)=CC4=NC5=CC(Cl)=CC6=NC(=C1)N2C(N34)N56 ; Clc1nc(Cl)c2nc(Cl)nc(Cl)c2n1]

In one embodiment, the invention includes compound comprising an alkyl-linked polyarylether network (PAE) formed by a plurality of alkyl-linked polyether compounds according to Formula (III):

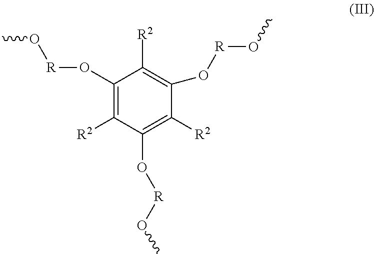

wherein R is independently alkyl or aryl, and R² is independently an electron withdrawing group. In another embodiment, R is a C₄₋₁₂ linear alkyl, and the electron withdrawing group is selected from: NO₂, CN, CHO, halogen, CO₂R³, CONR³, CH═NR³, (C═S)OR³, (C═O)SR³, CS₂R³, SO₂R³, SO₂NR³, SO₃R³, P(O)(OR³)₂, P(O)(R³)₂, or B(OR³)₃ type wherein R³ is an alkyl, an aryl or a hydrogen atom. In a preferred embodiment, the compound according to Formula (III) can include an electron withdrawing group selected from: CN, CHO, or halogen. In another preferred embodiment, the compound of Formula (II) can for a monomer unit that can form an alkyl-linked polyarylether network (PAE) as described herein generally.

Additional embodiments include methods of synthesizing an alkyl-linked polyarylether monomer/network comprising the steps according to the following scheme:

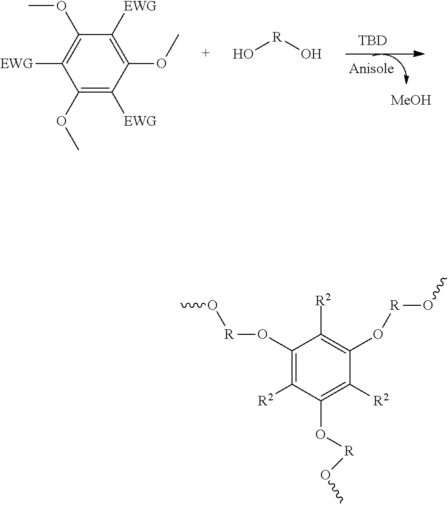

wherein R is independently alkyl or aryl, and R² is independently an electron withdrawing group as described herein.

Additional embodiments of the invention further include methods of synthesizing a polyarylether comprising the step of reacting a di/triarylether with two/three cyano groups and an alcohol through a nucleophilic aromatic substitution (S_{N}Ar) reaction. Additional embodiments of the invention further include methods of synthesizing polyarylether comprising the step of reacting a di/triarylether with two/three aldehyde groups and an alcohol through a nucleophilic aromatic substitution (S_{N}Ar) reaction. Additional embodiments of the invention further include methods of synthesizing synthesizing a polyarylether comprising the step of reacting a di/triarylether with two/three halogen groups and an alcohol through a nucleophilic aromatic substitution (S_{N}Ar) reaction.

In another preferred embodiment, the invention includes alkyl- and/or aryl-linked polycyanurate compound comprising:

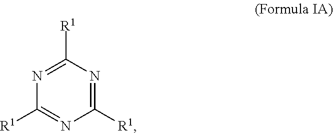

wherein R¹ is an alkyl or aryl diol. In a preferred embodiment, the alkyl diol of the compound of Formula IA comprises:

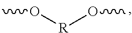

wherein R is a C₄₋₁₂ linear alkyl or an aromatic diol. In an alternative preferred embodiment, the alkyl diol of Formula IA is selected from the group consisting of: 1,4-butandiol, 1,6-hexandiol and 1,12-dodecanediol.

In another embodiment, the invention may include an alkyl-linked polycyanurate network (PCN formed by a plurality of alkyl-linked polycyanurate compounds according to Formula II:

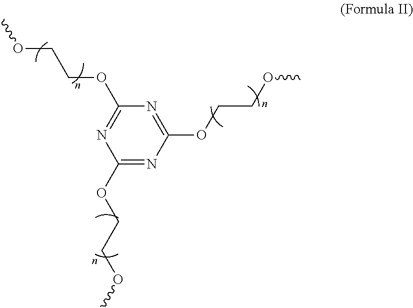

In this preferred embodiment, the n of Formula II may be between 2-6.

Additional embodiments of the invention include methods of synthesizing an alkyl-linked polycyanurate monomer/network comprising the steps according to the following scheme:

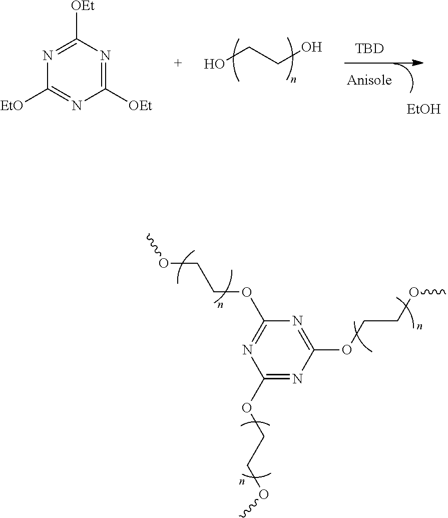

In this preferred embodiment, the n of the above method may be between 2-6.

Additional embodiments of the invention include methods of synthesizing an alkyl-linked polycyanurate from an aryl polycyanurate comprising the steps according to the following scheme:

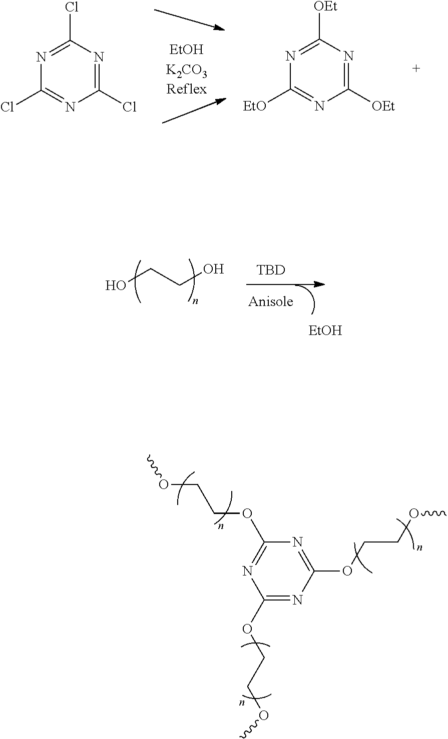

In this preferred embodiment, the n of the above method may be between 2-6.

Additional embodiments of the invention include methods of synthesizing an alkyl-linked polycyanurate comprising the step of forming a single bond between a triazine carbon and an oxygen through a nucleophilic aromatic substitution (S_{N}Ar) reaction.

Additional embodiments of the invention include methods of synthesizing an alkyl-linked polycyanurate comprising the step of reacting an alkyl cyanurate and an alcohol through a nucleophilic aromatic substitution (S_{N}Ar) reaction.

Additional embodiments of the invention include methods of synthesizing an alkyl-linked polycyanurate comprising the step of reacting 2,4,6-triethoxy-1,3,5-triazine (TETA) and an alcohol in the presence of triazabicyclodecene (TBD).

Additional embodiments of the invention include methods of converting an alkyl-lined PCNs into its monomer subunits comprises the step according to the following scheme:

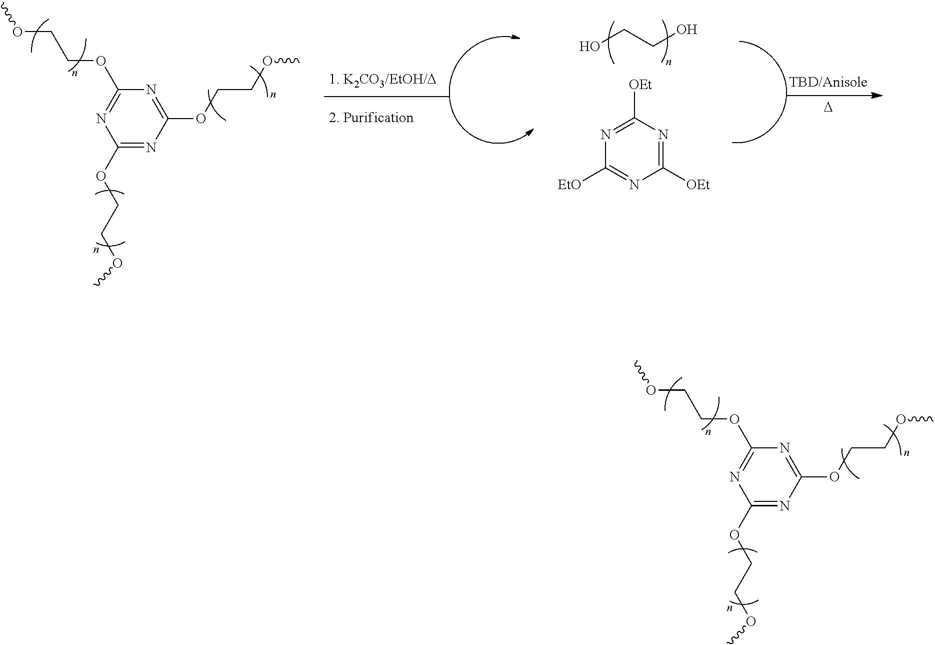

Additional embodiments of the invention include methods of upcycling an aryl-PCN to TETA and bisphenol A (BPA) according to the following scheme:

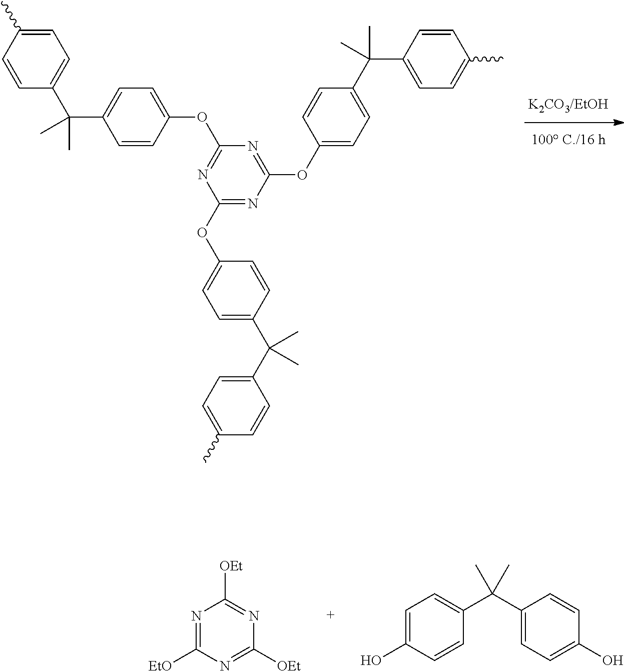

### Section 1. Materials and Instruments

Acetone (99.5%), dichloromethane (99.5%), ethanol (200 prof), methanol (99.8%), hexanes (98.5%), hydrogen peroxide (30%), hydrochloric acid (99.7%), sodium hydroxide (97.0%), tetrahydrofuran (99.9%), phenol (99%) and potassium carbonate anhydrous (99.0%) were purchased from Fisher Chemical. Cyanuric chloride (99%), 1,4-butanenaiol (99%), bisphenol A (99%), 1,4-dimethoxybenzne (99%), 1,12-dodecanediol (99%), sodium borohydride (97%) and 1,3,5-trimethoxybenzene (99.0%) were purchased from Sigma-Aldrich. Anisole (99.0%) and 1,6-hexanediol (97.0%) were purchased from TCI. Triazabicyclodecene (98%) and dicycanatobisphenol A (98%) were purchased from Combi-Blocks. p-cresol (98%) was purchased from Alfa Aesar. Deuterated chloroform (99.8%), deuterated methanol (99.8%) and deuterated benzene (99.5%) were purchased from Cambridge Isotope Laboratories. All chemicals were used directly without further purification. Polyethylene sample was obtained from Caplugs WW-9, polypropylene sample was obtained from Thermo Scientific centrifuge tube rack, polystyrene sample was obtained from Sigma-Aldrich polystyrene petri dish, and polysulfone sample was obtained from Cambro polysulfone container.

¹H-NMR and ¹³C-NMR spectra were obtained on a Bruker Avance-II 300M NMR Spectrometer. The chemical shift of the residual solvent signals C₆H₆, CHCl₃ or MeOH was used as the reference. Solid-state cross polarization magic angle spinning (CP/MAS) NMR spectra were recorded on a Varian INOVA 400 NMR spectrometer. Fourier transform Infrared (FT-IR) spectra were obtained on Agilent Cary 630 FTIR spectrometer. The high-resolution mass spectra were obtained on Waters SYNAPT G2 High Definition Mass Spectrometry System. Gas Chromatography-Mass Spectrometry (GC-MS) was acquired on Agilent 6890 Single quad GC mass spectrometer with Agilent VF-5-MS 30 m×0.25 mm×0.25 m column. The dynamic mechanical analysis (DMA) tests were performed on Q800 from TA Instruments. The differential scanning calorimetry (DSC) measurement was performed on Mettler Toledo DSC823. Ultraviolet-visible spectroscopy (UV-Vis) was measured on Agilent Cary 5000 UV-Vis-NIR. The moduli were measured from uniaxial tensile tests using Instron 5965. Thermogravimetric analyses (TGA) were performed on Thermogravametric Analysis Q500 from TA Instruments.

### Section 2. Monomer Synthesis and Characterizations

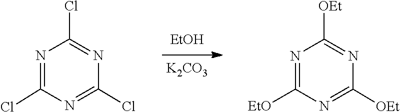

Preparation of 2,4,6-triethoxy-1,3,5-triazine (TETA) A suspension of cyanuric chloride (1.84 g, 10.0 mmol) and potassium carbonate (5.52 g, 40.0 mmol) in ethanol (40 mL) was heated in a Schlenk tube with stirring for 16 hours at 100° C. under nitrogen. Ethanol was removed via rotary evaporation after the suspension was cooled down to room temperature. Hexanes (60 mL) was added to the residue to dissolve the crude product. The mixture was filtered, and the solid was washed with hexanes (40 mL). The combined filtrate was washed with water (2×50 mL) and brine (50 mL) and dried over anhydrous Na₂SO₄. After evaporating the volatiles, the product was obtained as a white crystal (1.79 g, 84.0%): ¹H-NMR (300 MHz, CDCl₃) δ 4.41 (q, J=7.0 Hz, 2H), 1.37 (t, J=7.1 Hz, 3H); ¹³C-NMR (75 MHz, CDCl₃) δ 173.05, 64.35, 14.34; ESI-TOF MS Calcd. for C₉H₁₅N₃O₃ [M+H]⁺ 214.1192, found 214.1178.

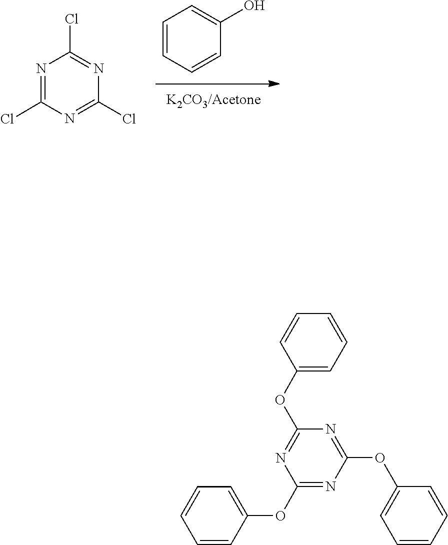 [SMILES: Clc1nc(Cl)nc(Cl)n1 ; Oc1ccccc1 ; c1ccc(Oc2nc(Oc3ccccc3)nc(Oc3ccccc3)n2)cc1]

Preparation of 2,4,6-triphenoxy-1,3,5-triazine (TPhTA) A suspension of cyanuric chloride (1.84 g, 10.0 mmol), phenol (4.70 g, 50.0 mmol) and potassium carbonate (6.90 g, 50.0 mmol), in acetone (40 mL) was heated in a Schlenk tube with stirring for 16 hours at 60° C. under nitrogen. Acetone was removed via rotary evaporation after the suspension was cooled to room temperature. The resulting solid was stirred in 1 M potassium carbonate solution (100 mL) for 10 minutes. The yellowish suspension was filtered and the off-while solid was washed with excessive water and methanol and dried in a vacuum oven at 60° C. to give pure TPhTA as a while solid (3.25 g, 91.0%): ¹H-NMR (300 MHz, CDCl₃) δ 7.43-7.29 (m, 6H), 7.25-7.19 (m, 3H), 7.17-7.08 (m, 6H). The data are consistent with the previous literature report¹.

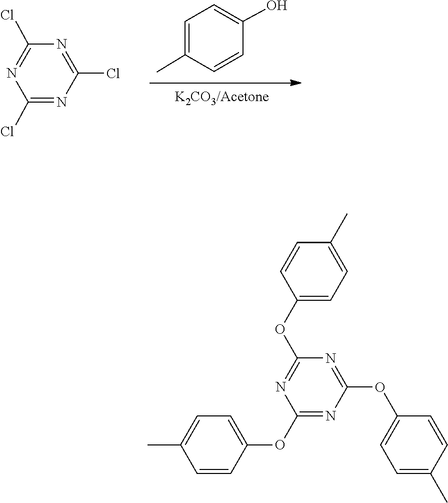

Preparation of 2,4,6-tris(p-tolyloxy)-1,3,5-triazine A suspension of cyanuric chloride (300 mg, 1.63 mmol), p-cresol (880 mg, 8.15 mmol), and potassium carbonate (1.12 g, 8.15 mmol) in acetone (15 mL) was heated in a Schlenk tube with stirring for 16 hours at 60° C. under nitrogen. Acetone was removed via rotary evaporation after the suspension was cooled down to room temperature. The resulting solid was stirred in 1 M potassium carbonate solution (20 mL) for 10 minutes. The yellowish suspension was filtered and the off-while solid was washed with excessive water and methanol and dried in a vacuum oven at 60° C. to give pure 2,4,6-tris(p-tolyloxy)-1,3,5-triazine as a while solid (3.25 g, 91.0%): ¹H-NMR (300 MHz, CDCl₃) δ 7.17-7.12 (m, 6H), 7.04-6.98 (m, 6H), 2.34 (s, 9H). The data are consistent with the previous literature report².

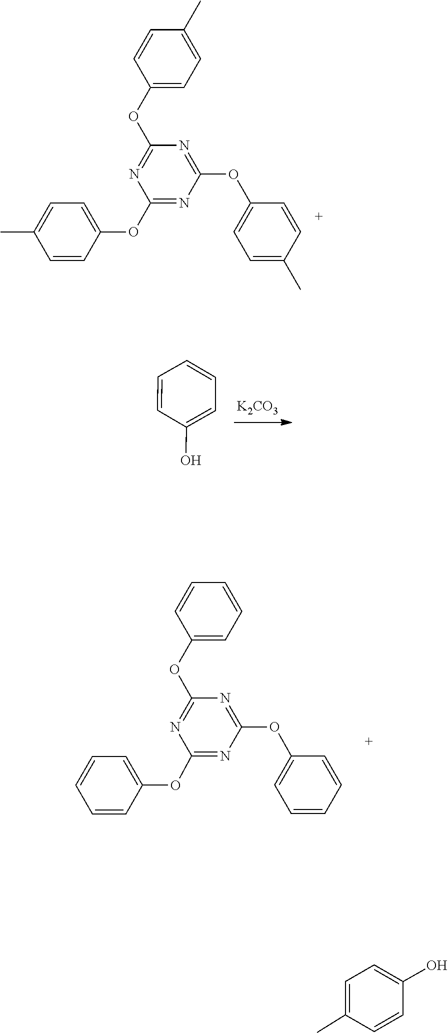

Converting 2,4,6-tris(p-tolyloxy)-1,3,5-triazine to TPhTA A suspension of 2,4,6-tris(p-tolyloxy)-1,3,5-triazine (40 mg, 0.100 mmol) and potassium carbonate (1.0 mg, 7.2 μmol) in ethanol (1.0 g) was heated in a 4 mL vial with stirring for 24 hours at 100° C. The mixture was cooled to room temperature and stirred in 1 M potassium carbonate solution (20 mL) for 10 minutes. The suspension was filtered and the off-while solid was washed with excessive 1 M potassium carbonate solution, water, and methanol and dried in a vacuum oven at 60° C. to give TPhTA (32 mg, 90%).

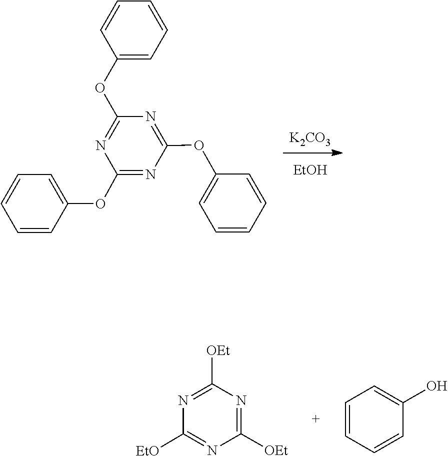

Converting TPhTA to TETA A suspension of TPhTA (107 mg, 0.300 mmol) and potassium carbonate (5.0 mg, 36 μmol) in ethanol (5.0 mL) was heated with stirring for 16 hours at 90° C. Ethanol was then removed via rotary evaporation. An aliquot of the resulting concentrate was analyzed by ¹H-NMR spectroscopy in 0.5 mL of CDCl₃. Nearly complete conversion of TPhTA to TETA and phenol was observed.

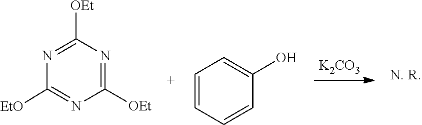

Reacting TETA with phenol TETA (107 mg, 0.500 mmol), potassium carbonate (5.0 mg, 36 mol), and phenol (94.1 mg, 1.00 mmol) were stirred at 90° C. for 16 hours. A ¹H-NMR spectrum of the mixture indicated that no reaction occurred.

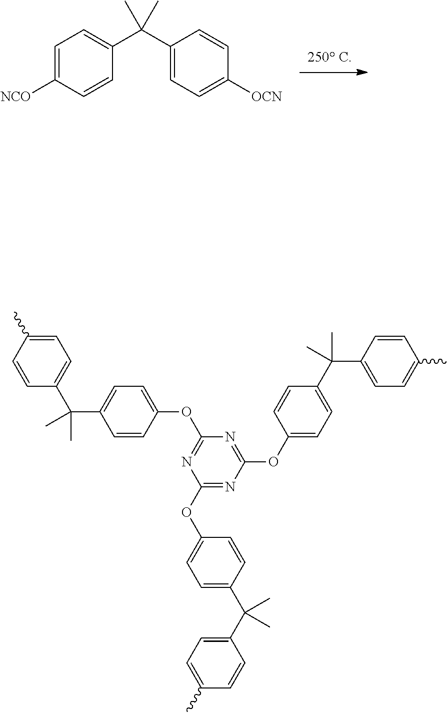

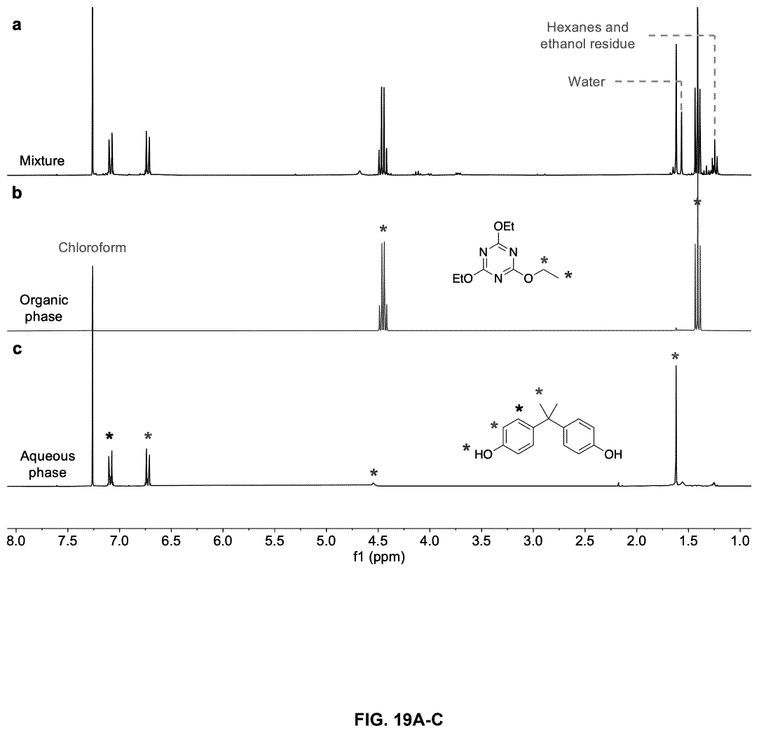

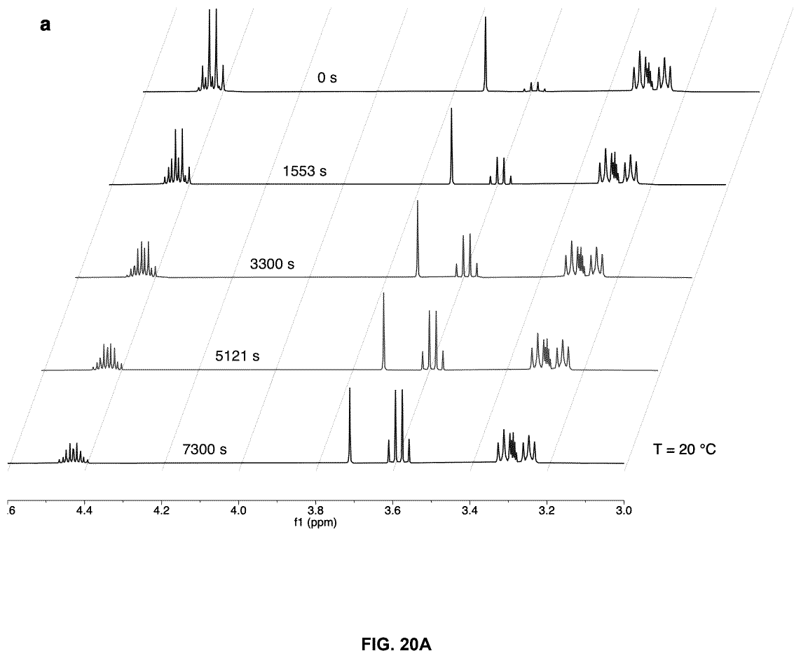

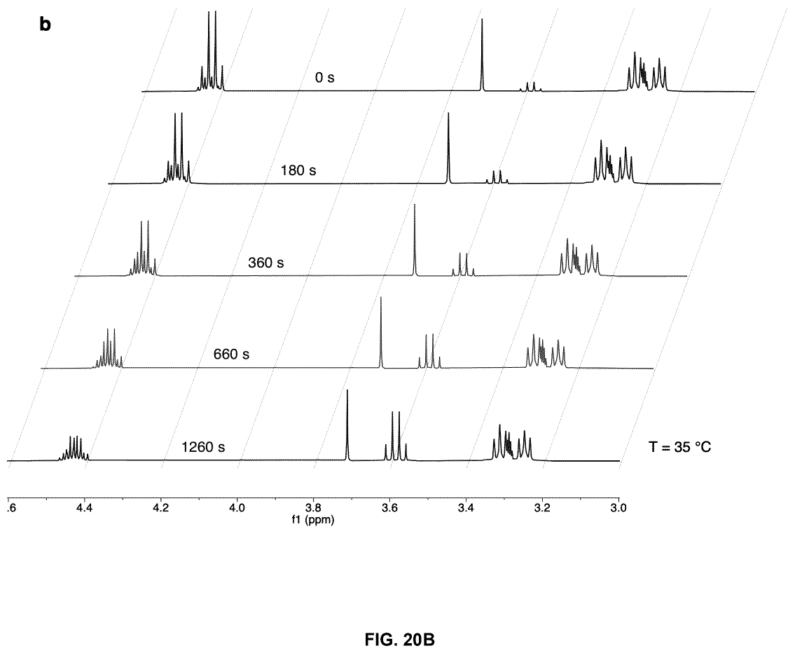

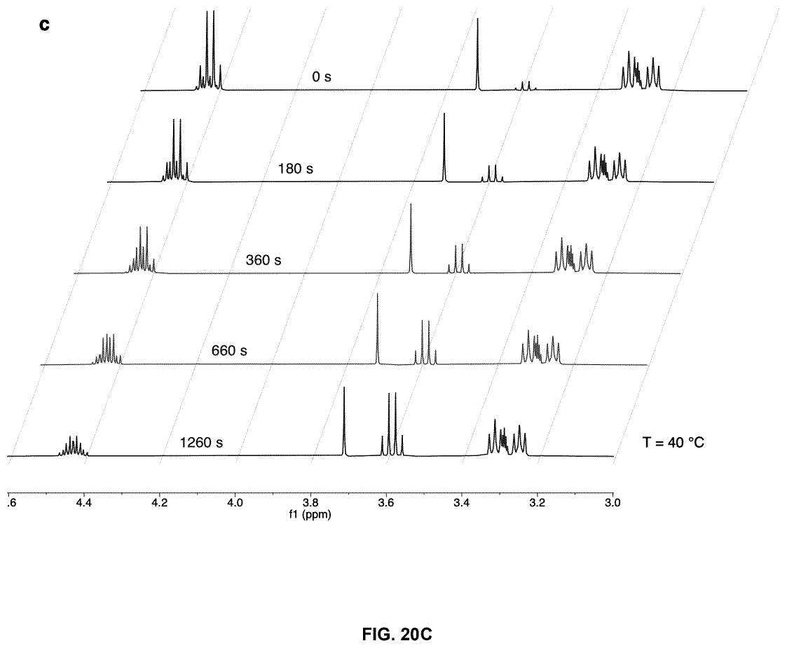

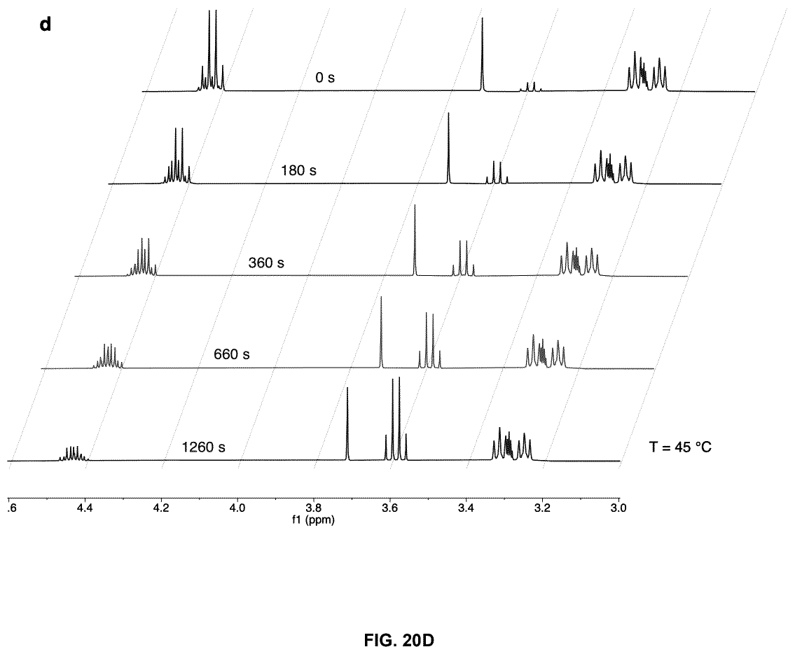

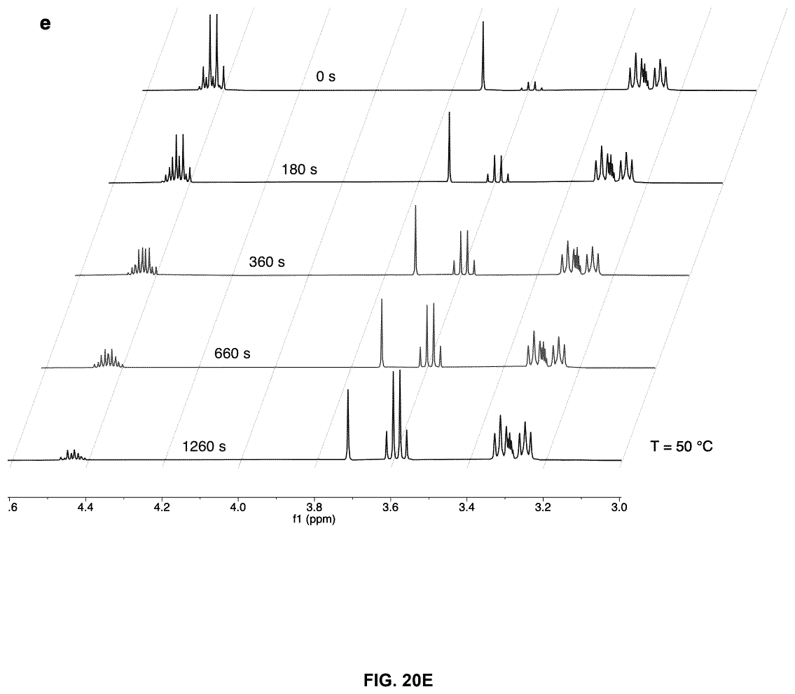

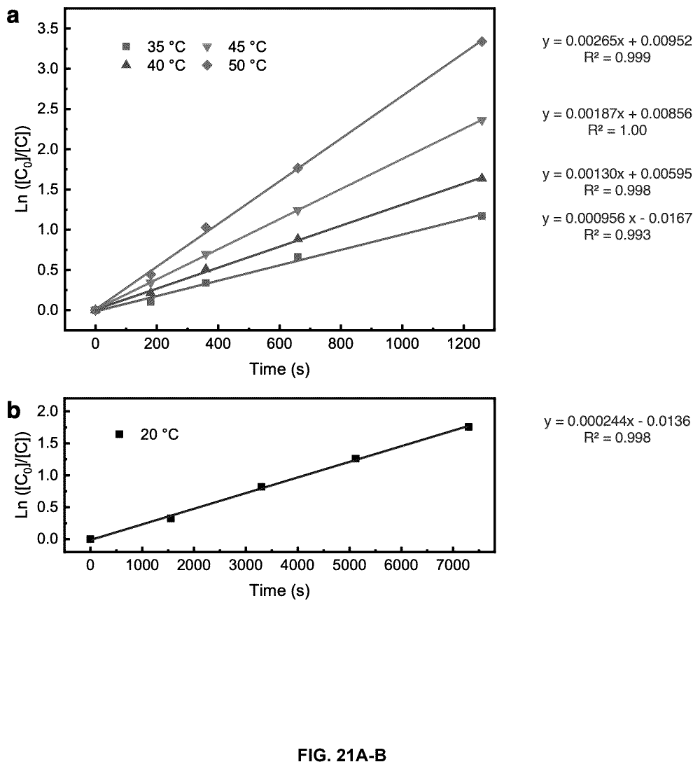

Preparing commercially available aryl PCN We chose commercially available cyanate resins Dicycanatobisphenol A (DCBPA) as the model system to investigate the possibility of upcycling PCN wastes to TETA monomer. DCBPA (2.0 g, 7.2 mmol) was weighted in an ampule. The ampule was cooled down to 77 k and sealed under vacuum (100 mTorr). After warming up to room temperature, the monomer was cured at 180° C. for 1 hour, 200° C. for 1 hour, 220° C. for 1 hour, 250° C. for 2 hours according to the literature.³ The product was obtained as translucent yellow solid. The FT-IR spectrum is consistent with the literature report, supporting the formation of PCN-DCBPA. c The obtained PCN-DCBPA were mechanically broken down to powdery solid before upcycling. A suspension of PCN-DCBPA (100 mg) and potassium carbonate (10.0 mg, 72.5 μmol) in ethanol (10 mL) was stirred at 100° C. for 16 hours. After cooling to room temperature, ethanol was removed by rotary evaporation. 0.1M NaOH solution (20 mL) and hexanes (20 mL) were added, and the mixture was sonicated for 5 minutes. The organic solution was separated, and the aqueous solution was extracted twice by hexanes (20 mL each). The organic solution was combined and washed by brine before being dried over anhydrous Na₂SO₄. After evaporating the volatiles, TETA was obtained as a white crystal (36.6 mg, 71%) (FIG. 19b). The aqueous phase was neutralized by 12M HCl before extracted by ethyl acetate (3×20 mL). The organic solution was combined and washed by brine before being dried over anhydrous Na₂SO₄. After evaporating the volatiles, Bisphenol A was obtained as a light yellow solid (72.3 mg, 88%) (FIG. 19c).

### Section 3. Small Molecule Model Reaction

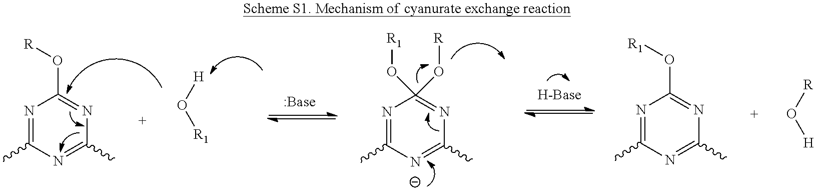

Thermodynamic equilibrium study for dynamic cyanurate exchange reaction TETA (128 mg, 0.601 mmol), methanol (58 mg, 1.80 mmol), and deuterated benzene (3.0 mL) were weighted in a 10 mL vial. The mixture was stirred at room temperature for 10 minutes. In NMR tube A was added 1 mL of the above solution. In NMR tube B was added 1 mL of the above solution together with TBD (1.7 mg, 12 μmol). The two NMR tubes were sealed and kept in the same oil bath at 60° C. The reactions in the two NMR tubes were monitored by ¹H NMR spectroscopy. There was no exchange reaction observed in tube A over the period of 24 hour. The reaction in the NMR tube B gradually reached the equilibrium after 40 hours.

Kinetics study with small molecules To deuterated methanol (3.96 g) was added TBD (41.2 mg, 29.6 μmol) and 1,3,5-trimethoxybenzene (8.4 mg, 50 μmol). The mixture was sealed and stirred at room temperature till a transparent solution was obtained. TETA (63.9 mg, 0.300 mmol) was added to the solution. The mixture was then sealed and stirred at room temperature for a few minutes to fully dissolve the TETA. The resulting solution was transferred to five NMR tubes (Trt, T35, T40, T45 and T50) and stored in ice bath to freeze the exchange reaction. Once Trt was submitted to ¹H-NMR measurement, T35, T40, T45, and T50 were simultaneously heated at 35° C., 40° C., 45° C., and 50° C., respectively. After 180 seconds, T35, T40, T45 and T50 were taken out of the oil bath and cooled in ice bath before submitted to ¹H-NMR spectra acquisition. This process was repeated to obtain the ¹H-NMR spectra of T35, T40, T45 and T50 at 180 s, 360 s, 660 s and 1260 s. ¹H-NMR spectra record times for Trt were 1553 s, 3300 s, 5121 s and 7300 s. The temperature at NMR facility was recorded as 20° C. Due to the slight decrease of electron donating effect of methoxy group, the —CH₂— on the remaining ethoxy groups have slightly higher chemical shift. Even though the —CH₂— signals of different species are overlapped, we can still use the integration of the far-right peak of the quartet on TETA (at 4.41 ppm) and the methoxy peak of 1,3,5-trimethoxybenzene (at 3.73 ppm) to quantify the amount of the unreacted TETA.

S_{N}Ar is formulated as a second-order reaction as shown in Equation S1. Since MeOH-d₄ was present in large excess and the proton exchange is much faster than the S_{N}Ar, the concentration of MeOH-d₄ was considered as a constant. Then Equation S1 can be simplified to Equation S2, where the experimental rate constant kₑₓₚ equals to the rate constant times the concentration of methanol. If the initial concentration of TETA was set to be [C]₀, Equation S2 can be further re-written as Equation S3.

$$\frac{d [ C ]}{dt} = - k [ C ] [ {CD}_{3} OD ] \qquad ( Eq . S 1 )$$

$$\frac{d [ C ]}{dt} = - {k}_{exp} [ C ] \qquad ( Eq . S 2 )$$

$$ln ( \frac{[ C ]}{{[ C ]}_{0}} ) = - {k}_{exp} t \qquad ( Eq . S 3 )$$

By plotting ln([C]₀/[C]) vs. time, the experimental rate constants under different temperatures were calculated and shown in Table 1.

TABLE 1
Experimental rate constants of cyanurate
exchange at different temperatures

| Temperature (° C.) | 20 | 35 | 40 | 45 | 50 |
|---|---|---|---|---|---|
| Rate Constant (10⁻⁵ L mol⁻¹ s⁻¹) | 0.988 | 3.87 | 5.26 | 7.45 | 10.7 |

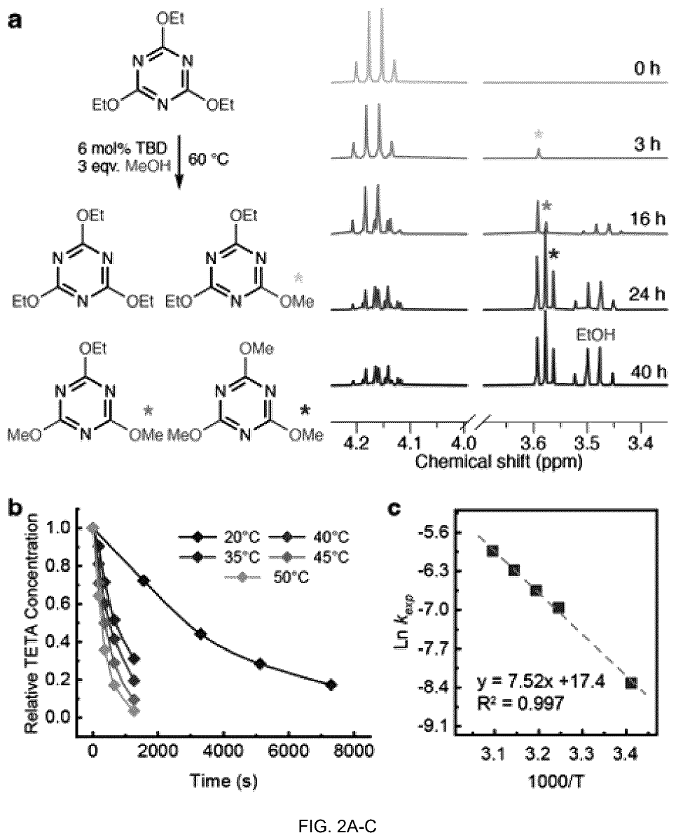

The Arrhenius equation (Equation S4) and its equivalent form (Equation S5) was used to calculate the reaction activation energy (Eₐ), wherein A and B are fitting parameters, and R is the gas constant, 8.31 J/(mol·K). By fitting with the experimental data in FIG. 2c, Eₐ was calculated to be 62.7 kJ/mol.

$$\frac{{k}_{exp}}{[ {DOCD}_{3} ]} = k = {Ae}^{- \frac{{E}_{a}}{RT}} \qquad ( Eq . S 4 )$$

$$ln {k}_{exp} = - \frac{{E}_{a}}{RT} + B \qquad ( Eq . S 5 )$$

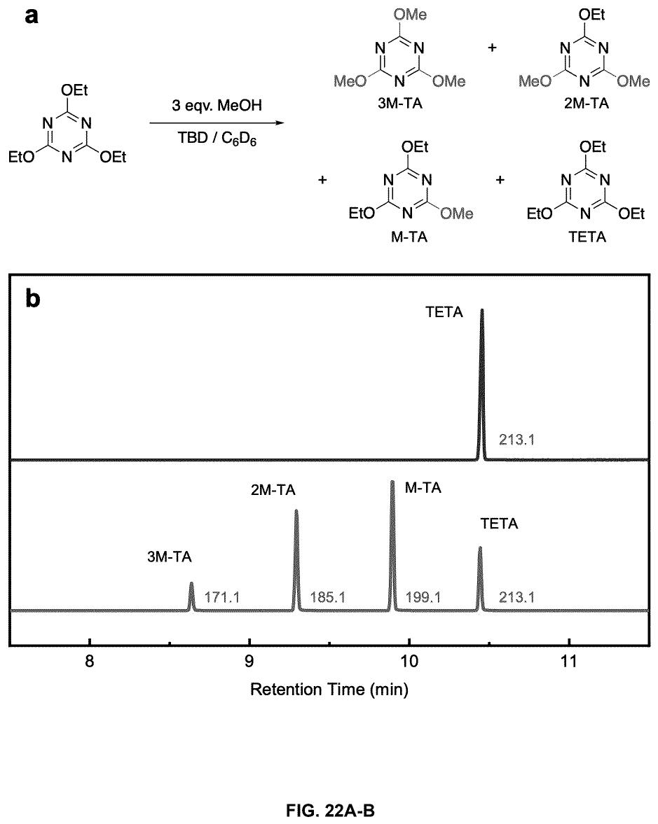

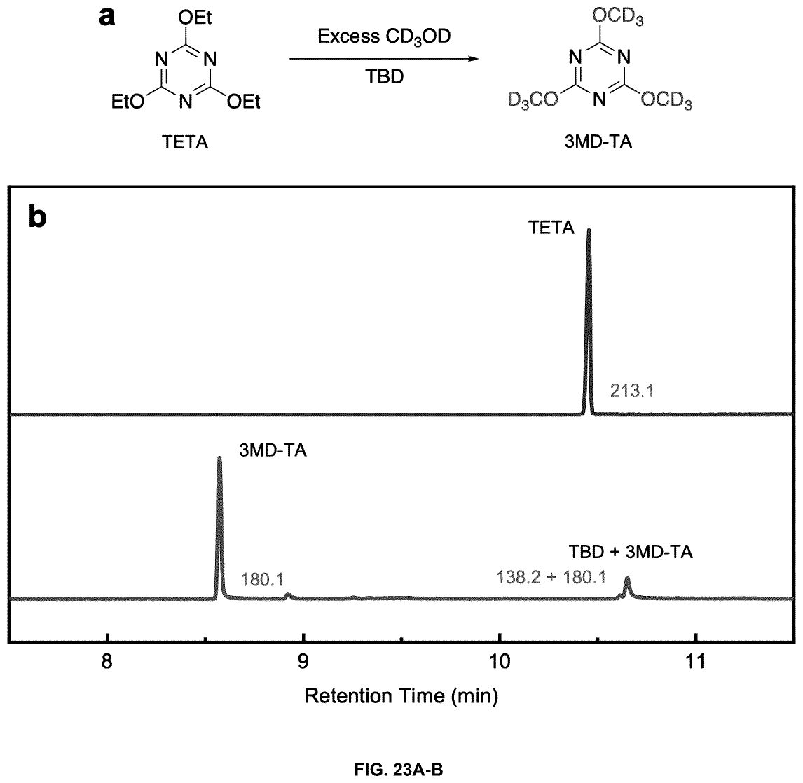

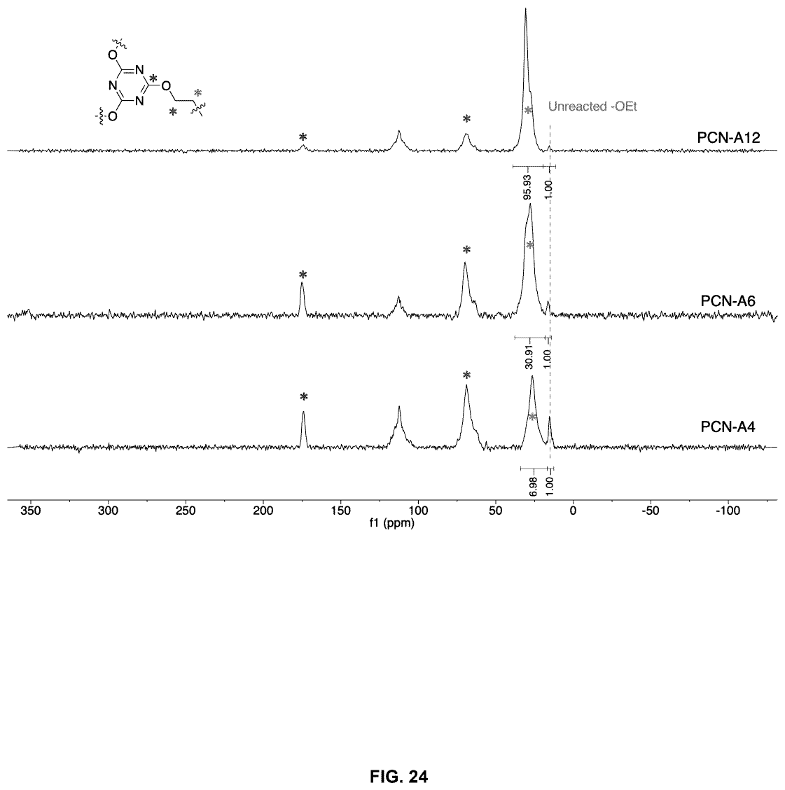

GC-MS measurements GC-MS tests were carried under nitrogen flow at 1 mL/min rate. Temperature was hold at 60° C. for 1.5 minutes, then 15° C./min ramp to 325° C. followed by holding at 325° C. for 1 minute. Three samples were analyzed by GC-MS in order to confirm the ¹H-NMR analysis for small molecule model reactions. we observed that after 40 hours of reaction with three equivalents of methanol, four different alkoxy-substituted triazines formed. According to the mass spectra, they are assigned to 3M-TA, 2M-TA, M-TA and TETA as shown in FIG. 22A-B. When TETA is reacted with large excessive of methanol, only one major peak was observed in GC. Mass spectrum indicates all TETA was converted into 3MD-TA as shown in FIG. 23. Therefore, the GC-MS results are consistent with the ¹H-NMR results shown above. Sample TETA preparation: TETA was dissolved in acetonitrile (1 mg/mL).

Preparation of sample from thermodynamic equilibrium study: The solution was taken from tube B after 40 hours of reaction, the solvent was evaporated under high vacuum then diluted in acetonitrile (1 mg/mL). Preparation of sample from kinetics study: The solution of T50 was heated overnight then directly diluted in acetonitrile (1 mg/mL).

### Section 4. Polymer Synthesis and Characterizations

PCN-A4 film preparation A mixture of TETA (853 mg, 4.00 mmol), 1,4-butanediol (DO-4) (541 mg, 6.00 mmol) and TBD (33.5 mg, 0.24 mmol) in anisole (3 mL) was stirred at 100° C. The mixture turned to a homogeneous solution after 15-minute stirring. The solution was then poured into a petri dish (diameter=5 cm). The solvent was allowed to slowly evaporate at 120° C. for 14 hours, leaving a transparent defect-free polycyanurate film. The film was further cured using a heat press at 130° C. under ambient pressure for 4 hours. A transparent film (926 mg) was obtained. Around 9.4% of ethoxy group was left unreacted.

PCN-A6 film preparation A mixture of TETA (853 mg, 4.00 mmol), 1,6-hexanediol (DO-6) (709 mg, 6.00 mmol) and TBD (33.5 mg, 0.24 mmol) in anisole (3 mL) was stirred at 100° C. The mixture turned to a homogeneous solution after 5-minute stirring. The solution was then poured into a petri dish (diameter=5 cm). The solvent was allowed to slowly evaporate at 120° C. for 14 hours, leaving a transparent defect-free PCN film. The film was further cured using a heat press at 130° C. under ambient pressure for 4 hours. A transparent film (1.106 g) was obtained. Around 5.4% of ethoxy group was left unreacted.

PCN-A12 film preparation A mixture of TETA (569 mg, 2.67 mmol), 1,12-dodecanediol (DO-12) (809 mg, 4.00 mmol) and TBD (22.2 mg, 0.16 mmol) in anisole (3 mL) was stirred at 100° C. The mixture turned to a homogeneous solution after 5-minute stirring. The solution was then poured into a petri dish (diameter=5 cm) and kept in oven. The solvent was allowed to slowly evaporate at 120° C. for 8 hours, leaving a transparent defect-free polycyanurate film. The film was further cured using a heat press at 130° C. under ambient pressure for 4 hours. A transparent film (1.049 g) was obtained. Around 4.3% of ethoxy group was left unreacted.

Tensile tests Samples were cut into approximately 3.5 mm×0.3 mm×20 mm strips from the original pad-shape PCNs. Tensile tests were performed at room temperature with a strain rate at 2.5%/min until fracture.

Dynamic mechanical analysis (DMA) tests Samples were cut into approximately 3 mm×0.3 mm×10 mm strips from the original pad-shape PCNs. The samples were equilibrated at −50° C. for 5 minutes. Then they were heated at a rate of 2° C./min at oscillation frequency of 1 Hz to 120° C. for PCN-A4 and 90° C. for PCN-A6 and PCN-12.

Differential scanning calorimetry (DSC) tests Samples were loaded into TZero Aluminum pans and scanned against an empty reference pan. After equilibration at −50° C., the temperature was ramped at 10° C./min to 160° C.

Gel fraction tests PCNs were cut into small pieces and submerged in different solvents (tetrahydrofuran, acetone, dichloromethane, and ethanol, 2 mL). After kept at room temperature for 48 hours, the solvents were decanted, and the residues were washed with an equal amount of the solvent for 5 times before dried in a vacuum oven at 60° C. for 4 hours. The weights before and after the test were recorded (Table 2).

TABLE 2
Gel fraction test results for PCN samples.

| | | Before | After | Before | After | Insoluble |
|---|---|---|---|---|---|---|
| Sample | Solvent | (mg) | (mg) | (mg) | (mg) | Fraction (%) |
| PCN-A4 | THF | 178.8 | 175.3 | 34.9 | 34.1 | 98.0 ± 0.42 |
| | Acetone | 101.0 | 100.6 | 39.8 | 39.2 | 99.1 ± 0.78 |
| | DCM | 108.2 | 107.5 | 28.4 | 27.7 | 98.3 ± 1.06 |
| | Ethanol | 116.7 | 116 | 30.1 | 29.9 | 99.2 ± 0.14 |
| PCN-A6 | THF | 186.9 | 185.1 | 40.1 | 39.6 | 98.9 ± 0.14 |
| | Acetone | 101.8 | 100.8 | 45.6 | 45.2 | 99.3 ± 0.21 |
| | DCM | 103.0 | 101.7 | 37.7 | 37.1 | 98.6 ± 0.28 |
| | Ethanol | 99.6 | 98.7 | 37.2 | 37.0 | 99.3 ± 0.28 |
| PCN-A12 | THF | 185.6 | 183.9 | 33.6 | 33.3 | 99.1 ± 0.0 |
| | Acetone | 104.6 | 103.7 | 36.5 | 36.1 | 99.2 ± 0.35 |
| | DCM | 102.2 | 101.6 | 36.8 | 36.6 | 99.3 ± 0.28 |
| | Ethanol | 100.3 | 99.1 | 34.7 | 34.7 | 99.4 ± 0.85 |

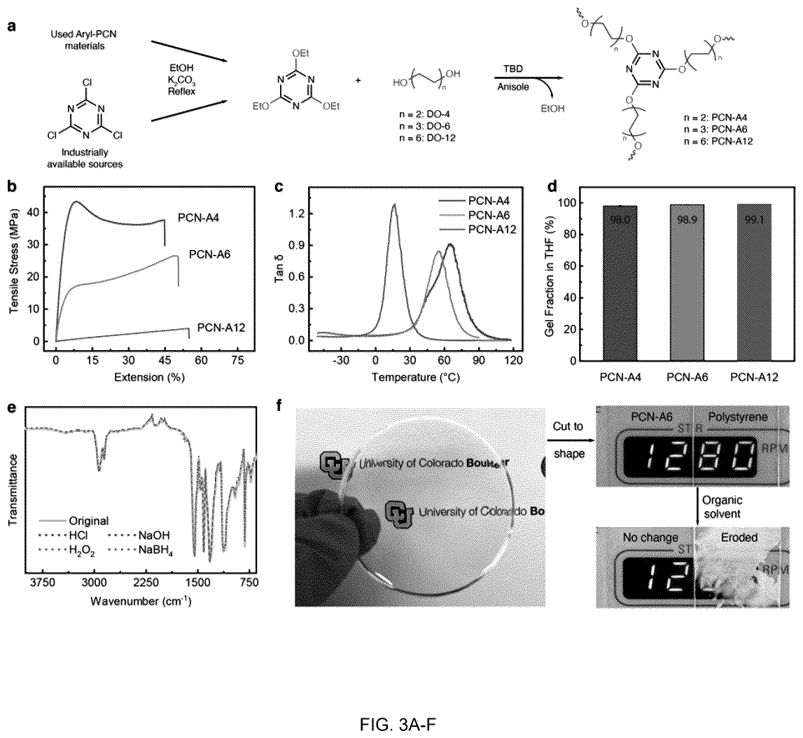

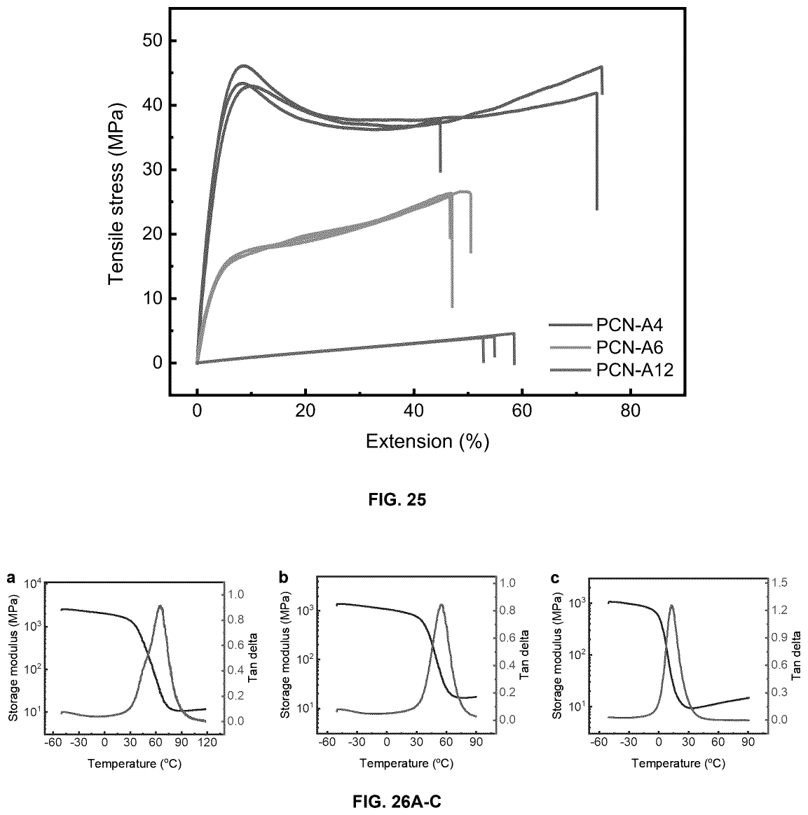

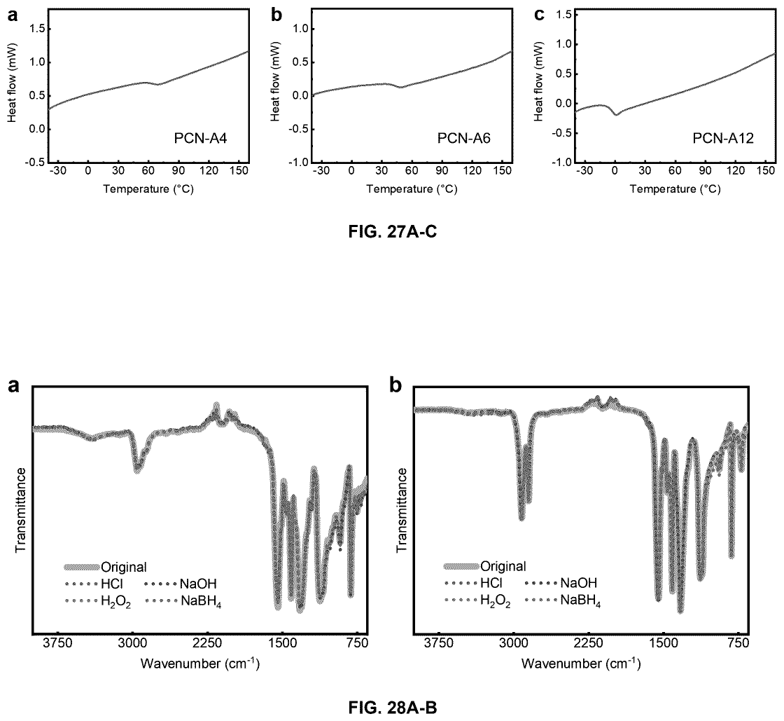

Chemical resistant tests PCNs were cut into small rectangular pieces (˜3 mg) and submerged in different solutions (1 N HCl, 1 N NaOH, 30 H₂O₂, and 1 M NaBH₄ in TIF, 2 mL). After kept at room temperature for 48 hours, the solutions were decanted, and the residues were washed with excessive water for 5 times and acetone for 3 times before dried in an oven at 120° C. for 4 hours. The weights before and after the test were recorded (Table 3). The optical images before and after the test were recorded in Extended Data FIG. 3. The FT-TR spectra before and after the test were shown in FIG. 3e and FIG. 25.

TABLE 3
Chemical resistance test results for PCN samples.

| | | Before | After | Before | After | Insoluble |
|---|---|---|---|---|---|---|
| Sample | Solution | (mg) | (mg) | (mg) | (mg) | Fraction (%) |
| PCN-A4 | 1N HCl | 28.7 | 28.2 | 29.9 | 29.6 | 98.6 ± 0.52 |
| | 1N NaOH | 29.9 | 29.4 | 25.9 | 25.2 | 97.8 ± 0.73 |
| | 30% H₂O₂ | 27.9 | 27.5 | 28.2 | 27.6 | 98.2 ± 0.49 |
| | 3M NaBH₄ | 20.9 | 20.8 | 33.3 | 33.0 | 99.3 ± 0.30 |
| PCN-A6 | 1N HCl | 33.7 | 33.4 | 22.5 | 22.4 | 99.3 ± 0.31 |
| | 1N NaOH | 33.6 | 33.3 | 26.9 | 26.3 | 98.4 ± 0.95 |
| | 30% H₂O₂ | 33.4 | 33.2 | 29.1 | 28.9 | 99.4 ± 0.06 |
| | 3M NaBH₄ | 25.9 | 25.7 | 29.7 | 29.4 | 99.1 ± 0.17 |
| PCN- | 1N HCl | 29.1 | 29.1 | 32.1 | 31.8 | 99.5 ± 0.66 |
| A12 | 1N NaOH | 36.9 | 36.8 | 28.1 | 27.8 | 99.3 ± 0.56 |
| | 30% H₂O₂ | 25.8 | 25.8 | 26.5 | 26.4 | 99.8 ± 0.27 |
| | 3M NaBH₄ | 35.0 | 34.6 | 29.6 | 29.5 | 99.3 ± 0.57 |

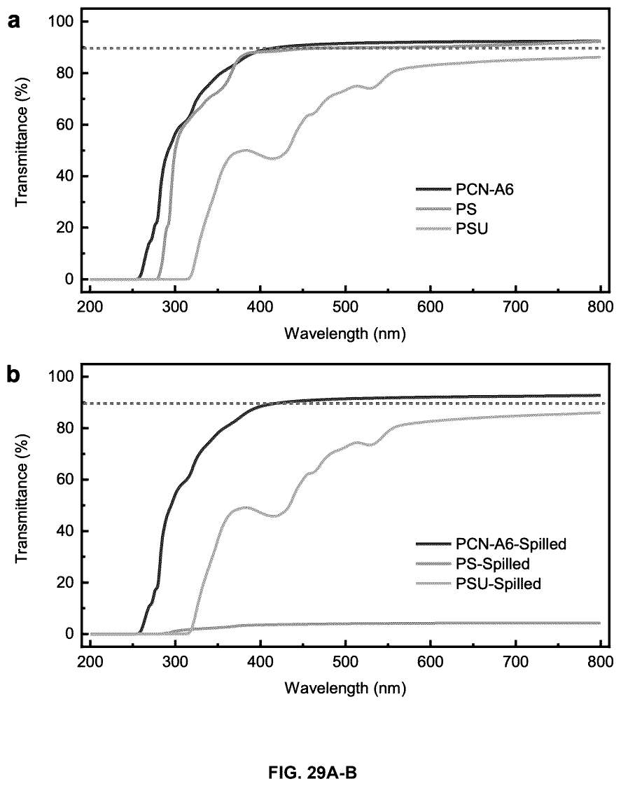

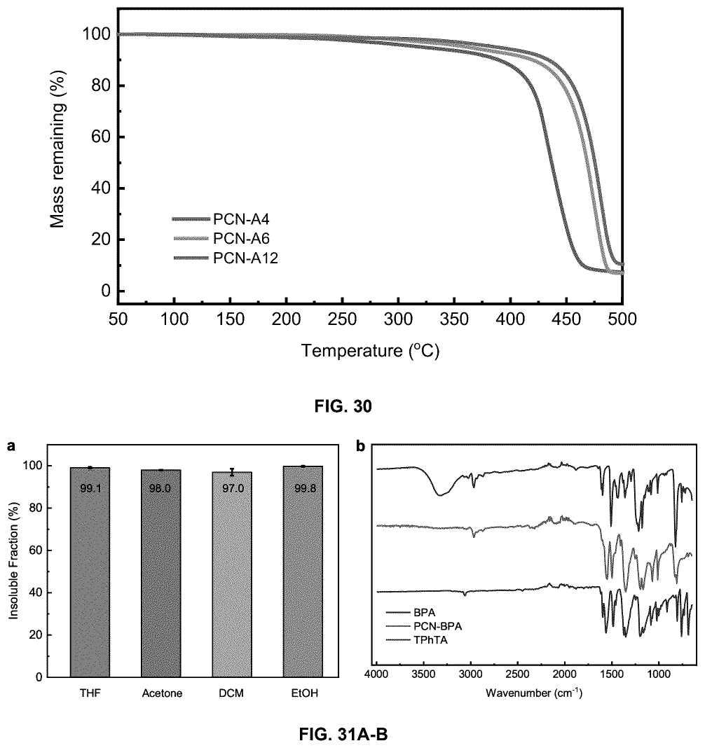

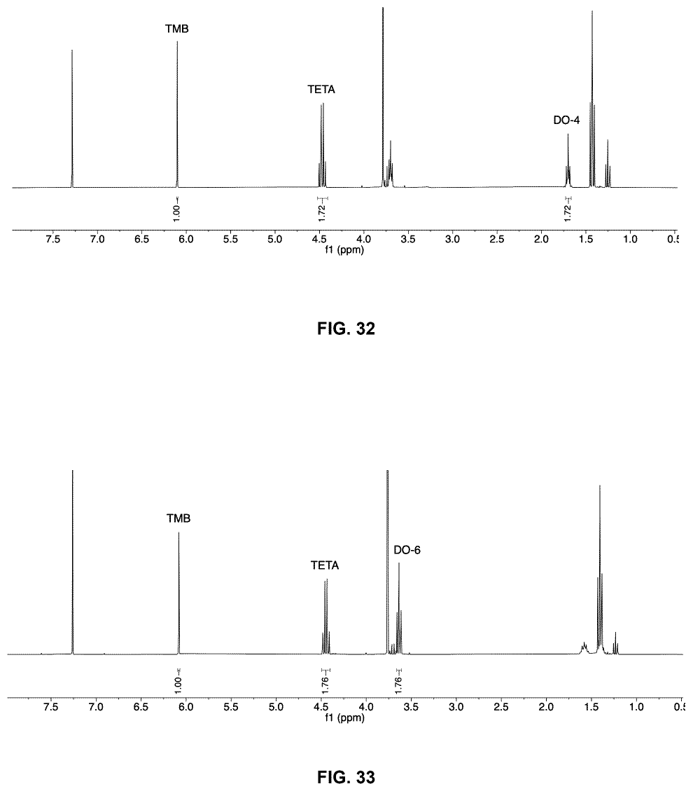

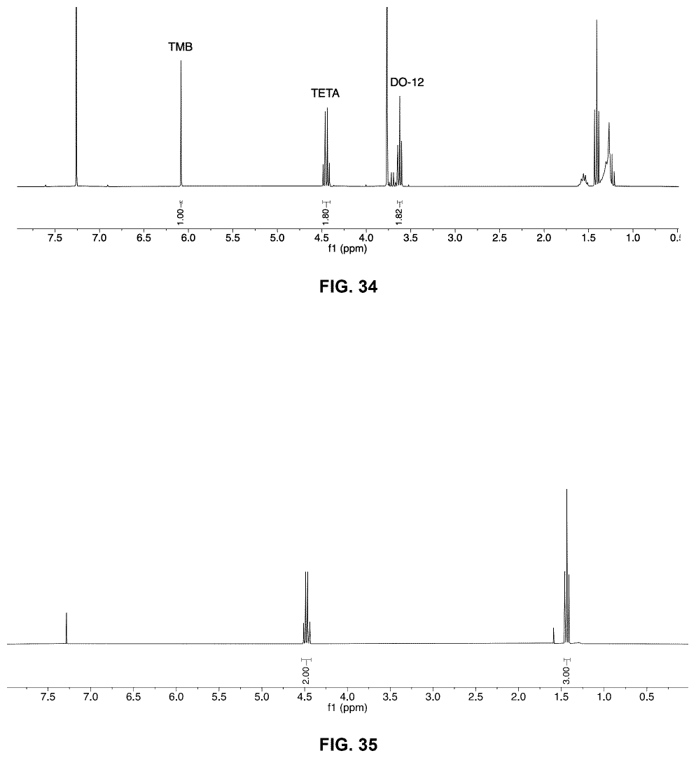

Solvent spill tests PCN-A6, polystyrene (PS) and polysulfone (PSU) were cut into rectangle shape (40 mm×30 mm) and their UV-Vis transmittance was measured. The plastics were then spilled with 0.5 mL of acetone, dichloromethane and ethanol, followed by UV-Vis transmittance measurements. As shown in FIG. 29, both virgin PCN-A6 and PS show over 90% transmittance in visible light area (400-800 nm). PSU shows decent transparency in long wavelength area but shows significant absorption below 550 nm, which is consistent with its amber color. Upon solvent spill, no noticeable change is observed in PCN-A6 and PSU, while there is a sharp drop in transmittance for PS. These results clearly demonstrate the excellent solvent resistance of PCN, which is similar to that of commercially available PSU.

Thermogravimetric analysis (TGA) tests Samples (˜3 mg) were tested with constant stream of nitrogen gas at temperature ramp of 10° C./min.

PCN-BPA synthesis A mixture of TPhTA (715 mg, 2.00 mmol), bisphenol A (685 mg, 3.00 mmol) and TBD (17 mg, 0.12 mmol) in 1,4-dimethoxybenzene (4.0 g) was stirred at 100° C. The mixture turned into a homogeneous solution after 15 minutes. The solution was then poured into a petri dish (diameter=5 cm). The solvent was allowed to slowly evaporate at 150° C. for 4 hours, 180° C. for 2 hours, 200° C. for 2 hours, 220° C. for 2 hours and 250° C. for 2 hours, leaving a yellowish transparent defect-free polycyanurate film. The appearance of aliphatic C—H absorption and disappearance of —OH absorption indicate formation of PCN-BPA⁴.

### Section 5: Chemical Recycling of PCNs

Degradation of PCN-A4 PCN-A4 (57.4 mg), potassium carbonate (3.0 mg, 22 μmol) and 1,3,5-trimethoxybenzene (TMB) (49.0 mg, 0.291 mmol) were weighed in a 10 mL vial. Ethanol (3 mL) was added. The mixture was treated as described in Methods for degradation procedure of the PCNs. The ¹H-NMR spectrum indicates TETA and diol are in 2:3 molar ratio. The original polymer contains around 8.9 mol % of unreacted —OEt group calculated by the mass of TMB.

Chemical recycling of TETA from PCN-A4 PCN-A4 (469 mg) and potassium carbonate (32.0 mg, 0.232 mmol) were stirred in ethanol (25 mL) at 90° C. for 16 hours. After the mixture was cooled to room temperature, the solid was filtered and washed with additional ethanol. Most of ethanol was removed via rotary evaporation, but not to dryness to prevent repolymerization. High vacuum was then applied at room temperature to remove the ethanol residue. Hexanes (10 mL) was added, and the mixture was sonicated for 5 minutes. The hexane solution was separated and DO-4 layer was washed with hexanes (5 mL) for two more times. The combined hexanes filtrate was washed with brine, dried over anhydrous Na₂SO₄, and concentrated to give the TETA as an off-white crystal (385 mg, 81% recovery yield).

Degradation of PCN-A6 PCN-A6 (52.7 mg), potassium carbonate (3.0 mg, 22 μmol) and 1,3,5-trimethoxybenzne (TMB) (37.7 mg, 0.224 mmol) were weighted to a 10 mL vial. Ethanol (3 mL) was added. The mixture was treated as described in Methods for degradation procedure of the PCNs. The ¹H-NMR spectrum indicates TETA and diol are in 2:3 mole ratio. The original polymer contains around 5.8 mol % of unreacted —OEt group calculated by the mass of TMB.

Chemical recycling of TETA from PCN-A6 PCN-A6 (524 mg) and potassium carbonate (30.0 mg. 0.217 mmol) were stirred in ethanol (25 mL) at 90° C. for 16 hours. The mixture was treated as described above for chemical recycling procedure of the PCN-A4. The TETA was recovered as white crystal (381 mg, 86% recovery yield).

Degradation of PCN-A12 PCN-A12 (66.5 mg), potassium carbonate (3.0 mg, 22 μmol) and 1,3,5-trimethoxybenzne (TMB) (31.4 mg, 0.187 mmol) were weighted to a 10 mL vial. Ethanol (3 mL) was added. The mixture was treated as described in Methods for degradation procedure of the PCNs. The ¹H-NMR spectrum indicates TETA and diol are in 2:3 mole ratio. The original polymer contains around 5.2 mol % of unreacted —OEt group calculated by the mass of TMB.

Chemical recycling of DO-12 and TETA from PCN-A12 PCN-A12 (506 mg) and potassium carbonate (25.0 mg, 0.181 mmol) were stirred in ethanol (25 mL) at 90° C. for 16 hours. After the mixture was cooled to room temperature, the solid was filtered and washed with additional ethanol. Most of ethanol was removed via rotary evaporation, but not to dryness to prevent repolymerization. High vacuum was then applied at room temperature to remove the ethanol residue. Hexanes (10 mL) was added, and the mixture was sonicated for 5 minutes. White solid precipitated out, then was filtered and washed with additional 20 mL of hexanes and water. The resulting white solid was dried under high vacuum to yield DO-12 as a white powder (385 mg, 95% recovery yield). The filtrate was transferred to a vial and all the volatiles were removed by rotary evaporation to give TETA as an off-white crystal (246 mg, 87% recovery yield).

Reformation of PCNs Procedures are the same as the PCNs synthesis but using recycled monomers. Defect-free transparent PCNs were obtained.

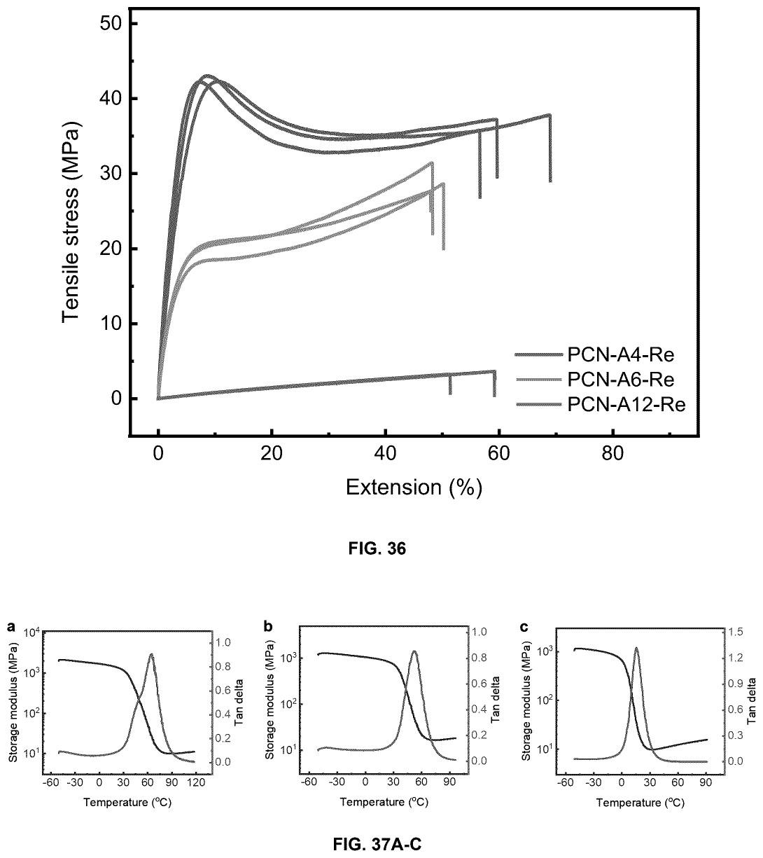

Characterization of reformed PCNs Tensile test and DMA test were performed. As shown in FIG. 36-37, no obvious chemical and mechanical property change compared to original PCNs was observed.

TABLE 4
Mechanical properties and T_{g} comparison of original and recycled PCNs

| | Young's Modulus (MPa) | | Tensile Stress (MPa) | | T_{g} (° C.) | |
|---|---|---|---|---|---|---|
| Sample | Original | Recycled | Original | Recycled | Original | Recycled |
| PCN-A4 | 1046 ± 69 | 1123 ± 91 | 44.9 ± 1.4 | 42.1 ± 1.0 | 65.3 | 65 |
| PCN-A6 | 188 ± 14 | 195 ± 14 | 25.9 ± 0.4 | 28.5 ± 1.4 | 54.5 | 53.0 |
| PCN-A12 | 8.47 ± 0.22 | 7.87 ± 0.39 | 4.07 ± 0.35 | 3.45 ± 0.20 | 16.0 | 15.5 |

### Section 6. Kinetics Studies of PCN-A6-m

Synthesis of PCN-A6-m PCN-A6-m preparation is the same as PCN-A6, but using 824 mg (3.86 mmol) of DO-6 and 201 mg (1.44 mmol) of TBD. In this case, ratio between alkoxy and free hydroxy is around 3:0.7. And the catalyst amount is 10 mol % to the alkoxy groups. The FTIR spectrum shows an obvious bump around 3400 cm⁴ for —OH.

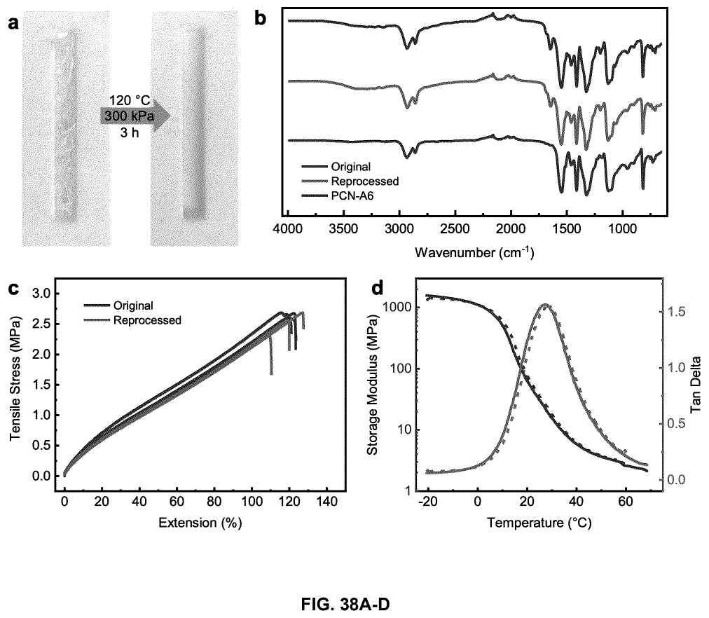

Reprocessing of PCN-A6-m Around 300 mg of PCN-A6-m was cut into small pieces that were used to fill the rectangular Teflon mold. The mold was heat pressed under 300 kPa and 120° C. for 3 hours and a defect-free film from recycled PCN-A6-m was obtained. The recycled films showed very similar FT-TR spectra, tensile strength and T_{g} to those of the virgin PCN-A6-m (FIG. 38 and Table 5).

TABLE 5
Mechanical properties comparison of
original and reprocessed PCN-A6-m.

| | Young's | Tensile | Elongation | T_{g} |
|---|---|---|---|---|
| Sample | Modulus (MPa) | Stress (MPa) | (%) | (° C.) |
| PCN-A6-m | 2.93 ± 0.24 | 2.64 ± 0.04 | 118 ± 3 | 27.4 |
| PCN-A6-m-Re | 2.66 ± 0.07 | 2.56 ± 0.06 | 119 ± 9 | 28.7 |

Stress relaxation tests PCN-A6-m samples were cut into approximately 3.5 mm×0.3 mm×6 mm strips. The sample was equilibrated at the set temperature for 30 min before pulling to 5% strain. The sample was allowed to relax to ˜₃₇% (1/e) of its original relaxation modulus at each temperature (110° C., 120° C., 130° C. and 140° C.).

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention. The term “stereoisomer” refers to a molecule that is an enantiomer, diastereomer or geometric isomer of a molecule. Stereoisomers, unlike structural isomers, do not differ with respect to the number and types of atoms in the molecule's structure but with respect to the spatial arrangement of the molecule's atoms. Examples of stereoisomers include the (+) and (−) forms of optically active molecules.

As used herein the singular forms “a”, “an”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth.

Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

As used herein, the term “electron withdrawing group” means a functional group having the ability to attract electrons, in particular if it is a substituent of an aromatic group, for example a group in particular of the NO2, CN, CHO, halogen, CO2R, CONR2, CH═NR, (C═S)OR, (C═O)SR, CS2R, SO2R, SO2NR2, SO3R, P(O)(OR)₂, P(O)(R)₂, or B(OR)₃ type wherein R is an alkyl, an aryl or a hydrogen atom.

The term “alkyl” refers to a saturated linear monovalent hydrocarbon moiety of one to twenty, typically one to fifteen, and often one to ten carbon atoms or a saturated branched monovalent hydrocarbon moiety of three to twenty, typically three to fifteen, and often three to ten carbon atoms. Exemplary alkyl group include, but are not limited to, methyl, ethyl, n-propyl, 2-propyl, tert-butyl, pentyl, iso-pentyl, hexyl, and the like.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group and are thus termed “fused aryl” or “fused aromatic”.

Aromatic groups containing one or more heteroatoms (typically N, O or S) as ring members can be referred to as heteroaryl or heteroaromatic groups. Typical heteroaromatic groups include monocyclic C5-C6 aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle”, “cycloheteroalkyl”, and “heterocyclic moieties” are used interchangeably herein and refer to any compound in which a plurality of atoms form a ring via a plurality of covalent bonds, wherein the ring includes at least one atom other than a carbon atom as a ring member. Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically, these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

As used herein, “alcohol” or “alcohols” refer to compounds having the general formula: R—OH, wherein R denotes any organic moiety (such as alkyl, aryl, or silyl groups), including those bearing heteroatom-containing substituent groups. In certain embodiments, R denotes alkyl, alkenyl, aryl, or alcohol groups. In certain embodiments, the term “alcohol” or “alcohols” may refer to a group of compounds with the general formula described above, wherein the compounds have different carbon lengths. As used herein, the term “alkanol” refers to alcohols where R is an alkyl group. In one preferred embodiment, alkyl PCNs may be prepared using an alcohol, such as 1,4-butandiol (DO-4), 1,6-hexandiol (DO-6) and 1,12-dodecanediol (DO-12) as the linkers through S_{N}Ar reaction (FIG. 3a).

The term “alkoxy” as used herein refers to a hydrocarbon group connected through an oxygen atom, e.g., —O-Hc, wherein the hydrocarbon portion He may have any number of carbon atoms, typically 1-10 carbon atoms, may further include a double or triple bond and may include one or two oxygen, sulfur or nitrogen atoms in the alkyl chains, and can be substituted with aryl, heteroaryl, cycloalkyl, and/or heterocyclyl groups. For example, suitable alkoxy groups include methoxy, ethoxy, propyloxy, isopropoxy, methoxyethoxy, benzyloxy, allyloxy, and the like.

“Cyanate ester resin” means a bisphenol or polyphenol, e.g. novolac, derivative, in which the hydrogen atom of the phenolic OH group is substituted by a cyano group, resulting in an —OCN group. Examples include but are not limited to bisphenol A dicyanate ester, commercially available as, e.g. Primaset® BADCy from Lonza or AroCy® B-10 from Huntsman, as well as other Primaset® or AroCy® types, e.g. bis(3,5-dimethyl-4-cyanatophenyl)methane (AroCy® M-10), 1,1-bis(4-cyanatophenyl)ethane (AroCy® L-10), 2,2-bis(4-cyanatophenyl)-1,1,1,3,3,3-hexafluoropropane (AroCy® F-10), 1,3-bis(1-(4-cyanatophenyl)-1-methylethylidene)benzene (AroCy® XU-366), di(4-cyanatophenyl)thioether (AroCy® RDX-80371; AroCy® T-10), bis(4-cyanatophenyl)dichloromethylidenemethane (AroCy® RD98-228), bis(4-cyanatophenyl)octahydro-4,7-methanoindene (AroCy® XU-71787.02L), as well as bis(4-cyanatophenyl)methane, bis(3-methyl-4-cyanatophenyl)methane, bis(3-ethyl-4-cyanatophenyl)methane, di(4-cyanatophenyl)ether, 4,4-dicyanatobiphenyl, 1,4-bis(1-(4-cyanatophenyl)-1-methylethylidene)benzene, and resorcinol dicyanate. See, also e.g., U.S. Pat. No. 10,233,139 to Evonik Technochemie GmbH.

As used herein, “triazines” refers to nitrogen-containing heterocycles. More particularly, “triazines” refers to six-membered rings having three carbon atoms and three nitrogen atoms as ring members. “Triazines” is intended to include substituted triazines or triazine derivatives, with melamines or aminoplasts being particularly preferred triazines for use as the first monomer in the inventive polymer.

As used herein, the term a “diol” refers to a chemical compound containing two hydroxyl groups (—OH groups).

As used herein, the term “thermoset” refers to a is a polymer that is obtained by irreversibly hardening (“curing”) a soft solid or viscous liquid prepolymer (resin).

The term “substituted” as used herein refers to a replacement of a hydrogen atom of the unsubstituted group with a functional group, and particularly contemplated functional groups include nucleophilic groups (e.g., NH2, OH, SH, CN, etc.), electrophilic groups (e.g., C(O)OR, C(X)OH, etc.), polar groups (e.g., OH), non-polar groups (e.g., heterocycle, aryl, alkyl, alkenyl, alkynyl, etc.), ionic groups (e.g., NH3+), and halogens (e.g., F, Cl), NHCOR, NHCONH2, OCH2COOH, OCH2CONH2, OCH2CONHR, NHCH2COOH, NHCH2CONH2, NHSO2R, OCH2-heterocycles, PO3H, SO3H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent. It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Dynamic Nucleophilic Aromatic Substitution Study in Small Model Compounds

S_{N}Ar (nucleophilic aromatic substitutions) on heterocyclic substrates such as pyridines, pyrimidines, and triazines, have been widely performed in medicinal chemistry. However, the application of such reactions in polymer synthesis has been much less explored. Although the bond exchange between phenols and aryloxy substituted triazines has been shown before, the dynamic S_{N}Ar reaction between alcohols and cyanurates has never been reported. In this context, at first, the presented used 2,4,6-triethoxy-1,3,5-triazine (TETA) and methanol as the model compounds to study the reversibility of S_{N}Ar between alkyl cyanurates and alcohols. The present inventors first demonstrated the thermodynamic equilibrium of cyanurate exchange reactions (FIG. 2a). In the absence of any catalyst, there is no exchange reaction between TETA and methanol at 60° C. (FIG. 6). By contrast, when a catalytic amount of triazabicyclodecene (TBD) was added, the exchange of ethoxy with methoxy groups occurred immediately, indicating the reversibility of such S_{N}Ar reaction. A diluted solution of TETA and methanol in 1:3 molar ratio provided four types of triazines in approximately 1:3:3:1 molar ratio after heating at 60° C. for 40 hours, where up to three ethoxy groups were replaced by methoxy groups. Such result indicates all three ethoxy groups are reactive and the exchange reaction has reached an equilibrium.

The kinetics of the cyanurate exchange was investigated at various reaction temperatures (FIG. 6). The reaction progress was monitored by measuring the intensity decrease of TETA proton signals in ¹H-NMR spectra over time. The S_{N}Ar reactions on 1,3,5-triazine core in phenolysis or aminolysis have been studied and a concerted second-order substitution mechanism was proposed. Considering a large excessive of methanol and fast proton transfer between the alcohol and TBD, the present inventors assumed the reaction is an irreversible pseudo-first-order reaction and calculated the experimental rate constants based on the TETA concentration decrease (FIG. 2b and Table 1). Using the reaction rate constants measured at different temperatures, the activation energy of the exchange reaction was calculated to be 62.5 kJ/mol from the Arrhenius plot and its linear fitting (FIG. 2c).

### Example 2: PCN Synthesis and Characterizations

Unlike well-known aryl PCNs, alkyl PCNs have been inaccessible because alkyl cyanate monomers undergo undesired isomerization to isocyanates under the conventional [2+2+2] trimerization conditions. There has been no viable synthetic approach for alkyl PCNs, which could have high impact-toughness compared to conventional aryl PCNs. The present inventors envisioned that the approach of the invention could provide alkyl PCNs with various thermal and mechanical properties, which have been challenging to achieve by using the traditional cyclotrimerization approach.

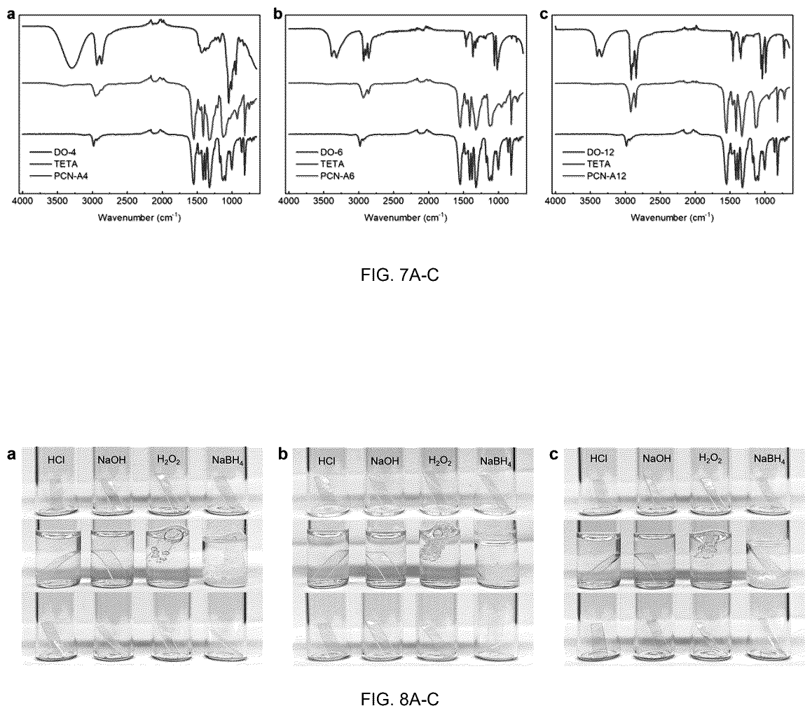

Thus, synthesis of alkyl PCNs using dynamic S_{N}Ar reaction of triazine with various diols was explored. Various alkyl PCNs were prepared using 1,4-butandiol (DO-4), 1,6-hexandiol (DO-6) and 1,12-dodecanediol (DO-12) as the linkers through S_{N}Ar reaction (FIG. 3a). TBD (2 mol % to the cyanurate group) was added as the catalyst. Fourier-transform infrared (FT-IR) spectra of the PCNs show the existence of C—O—C stretch band at 1130 cm⁻¹ and the triazine bands at 815 cm⁻¹ and 1550 cm⁻¹, and the disappearance of methyl rock band at 725 cm⁻¹ (FIG. 7), supporting the cyanurate structure with the ethoxy group replaced by alkyl diols. Solid-state cross polarization magic angle spinning (CP/MAS) NMR spectra show that only a small amount (4-10 mol %) of ethoxy groups remained unreacted. Thermogravimetric analysis (TGA) shows <4 wt % of mass loss below 300° C., which indicates high thermal stability of the polymer and the absence of volatile small molecule residue.

The mechanical properties of the PCNs were measured by the uniaxial tensile method. PCN-A4 showed elongation at the break over 45%, tensile strength of 45 MPa, and Young's modulus of 1.1 GPa, which is very ductile compared to common brittle aryl PCNs (˜5%, ˜90 MPa and ˜3.1 GPa in each value). With the increase of structural flexibilities of hydrocarbon chains between triazine nodes, the PCNs become softer and more ductile (FIG. 3b). A broad range of glass transition temperatures (T_{g}) could be obtained ranging from 15.5° C. for PCN-A12 to 54.5° C. and 65.5° C. respectively for PCN-A6 and PCN-A4 (FIG. 3c). The alkyl PCNs show high resistance to organic solvents. The gel fractions in various solvents measured by solvent extraction method were ˜99% for all three PCNs, supporting their highly crosslinked structures (FIG. 3d and Table 2).

Chemical resistance tests were also performed. After being kept under acidic (iN HCl), basic (1N NaOH), oxidative (30% H₂O₂), and reductive (1M NaBH₄ in THF) conditions for 48 hours, the PCNs retained almost identical appearances, weights, and chemical structures evidenced by FT-IR spectra, which indicates that PCNs are highly resistant to various chemical erosions (FIG. 3e and FIG. 8). Thus, the alkyl PCNs can be used as protective panels that provide high transparency and solvent/chemical resistance. The transparent PCN-A6 was cut into a rectangular shape and used to cover a digital display (FIG. 3f). Upon direct contact with common organic solvents (e.g., acetone, dichloromethane and ethanol), the film showed no change in shape or transparency, which is similar to polysulfone. On the contrary, the transparent polystyrene film was severely eroded. It should be noted that such S_{N}Ar approach can also provide aryl PCNs. As an example, PCN-DCBPA was successfully prepared via the condensation between 2,4,6-triphenoxy-1,3,5-triazine and bisphenol A (BPA).

### Example 3: Closed-Loop Recycling

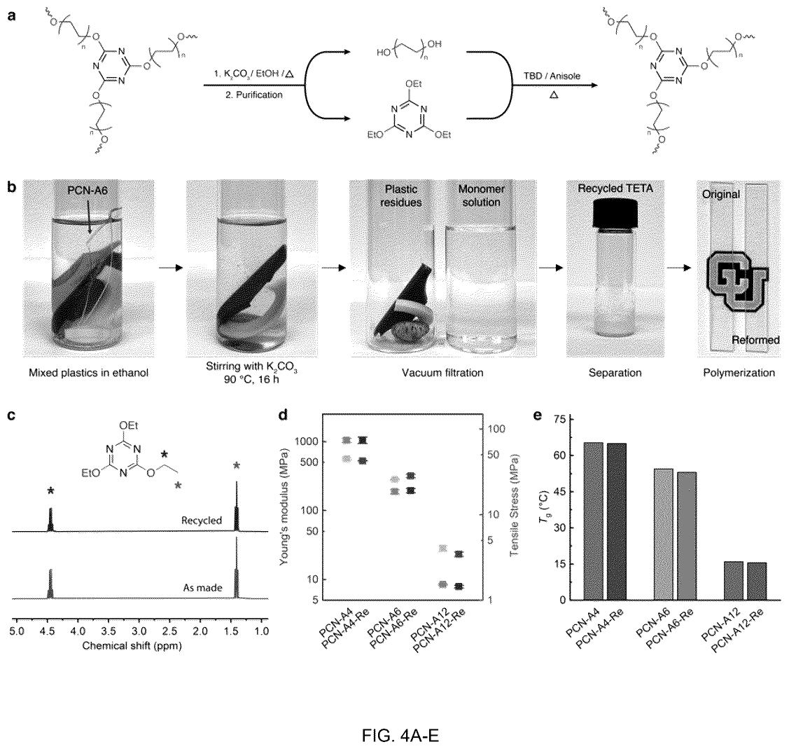

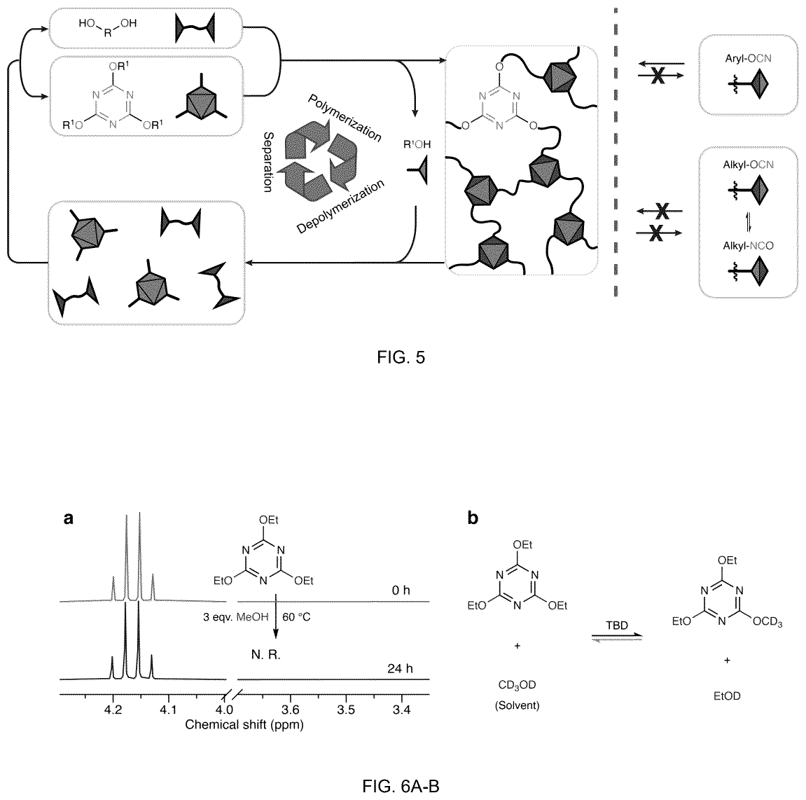

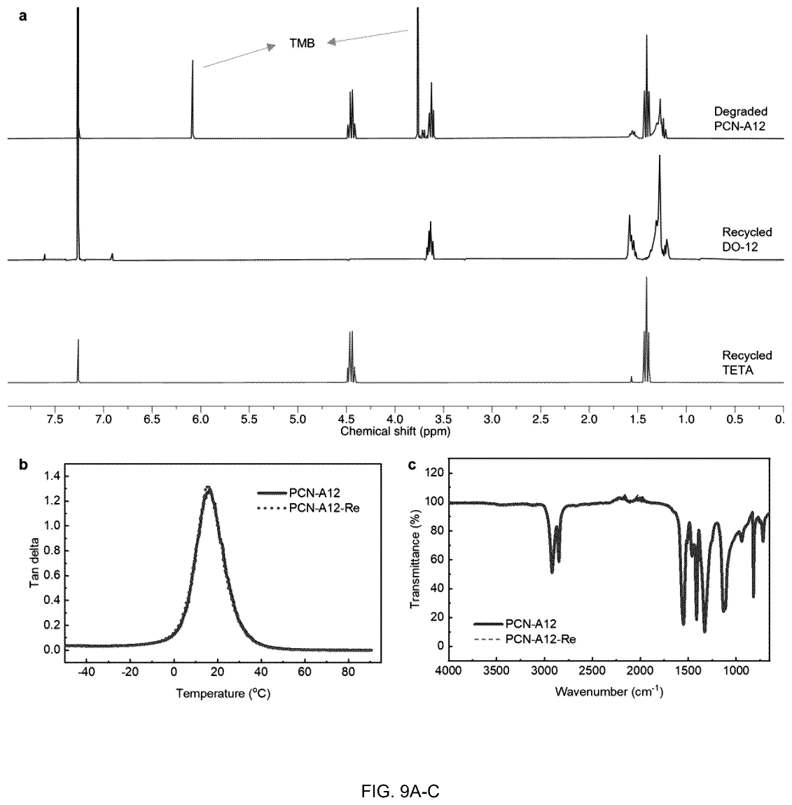

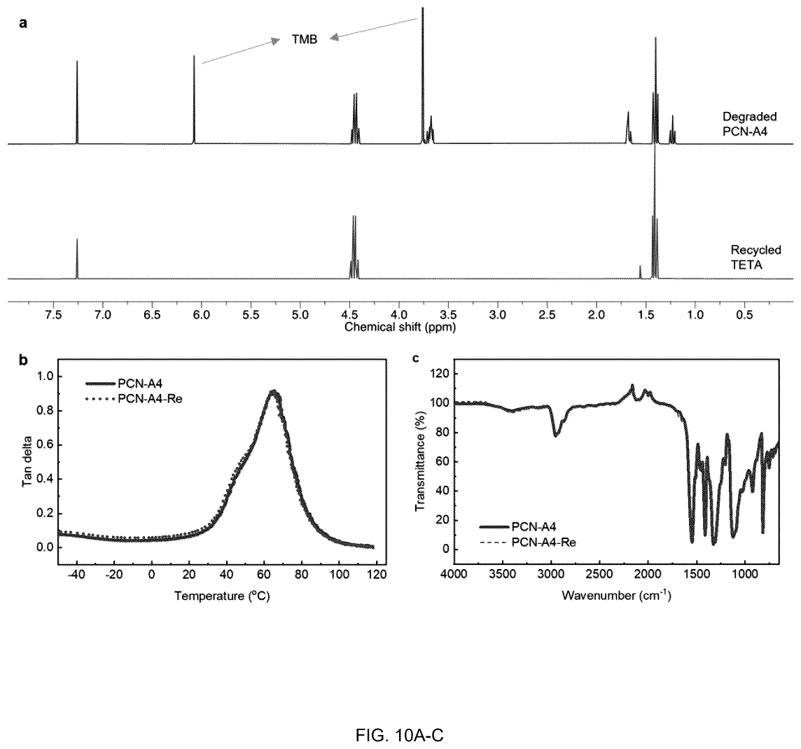

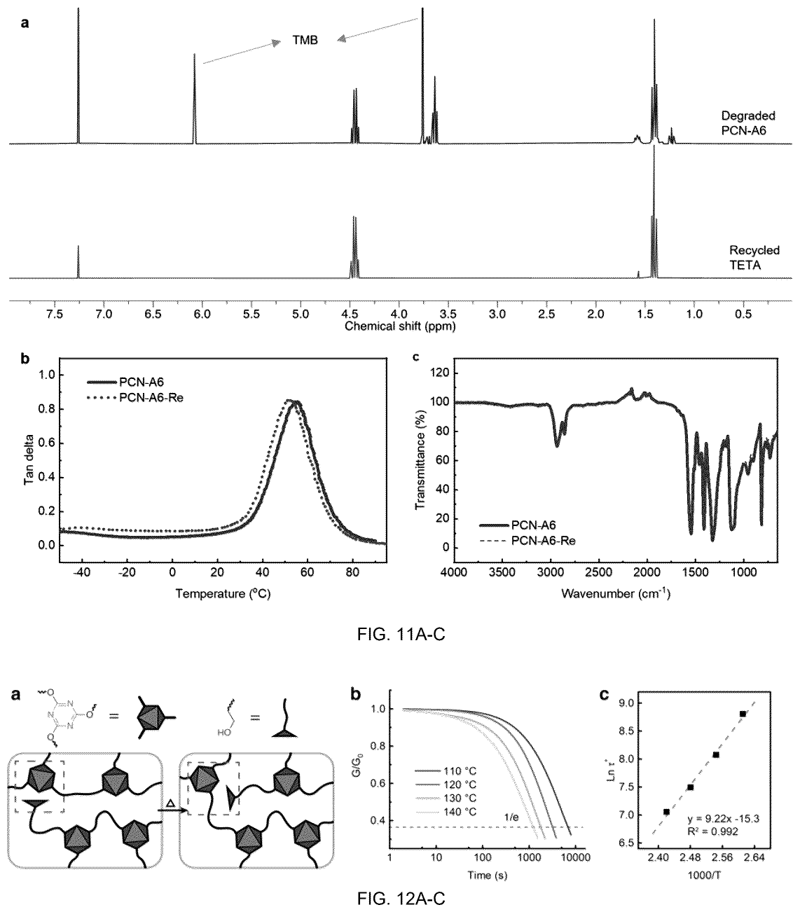

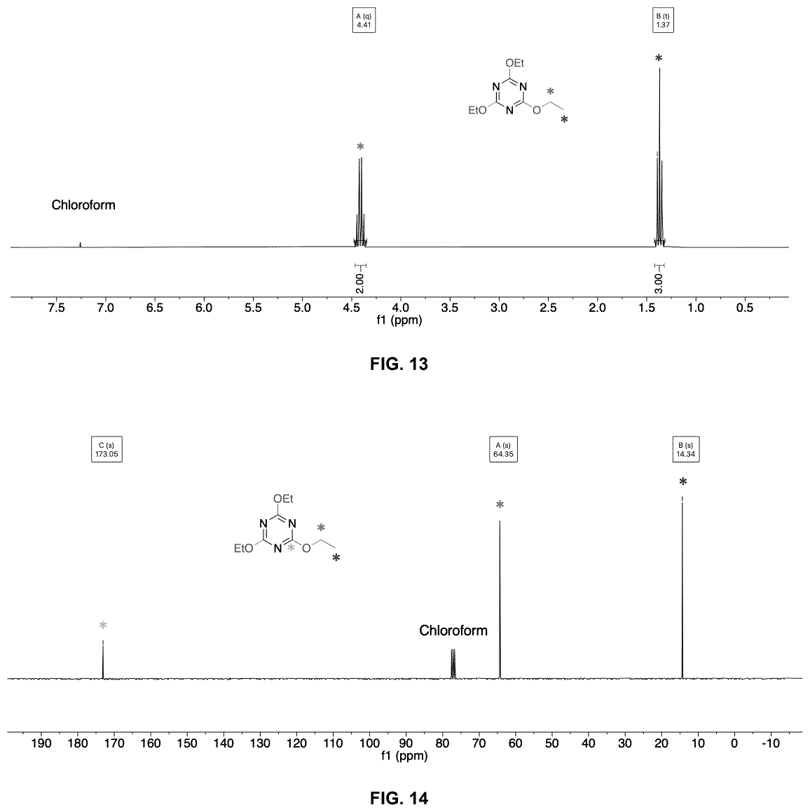

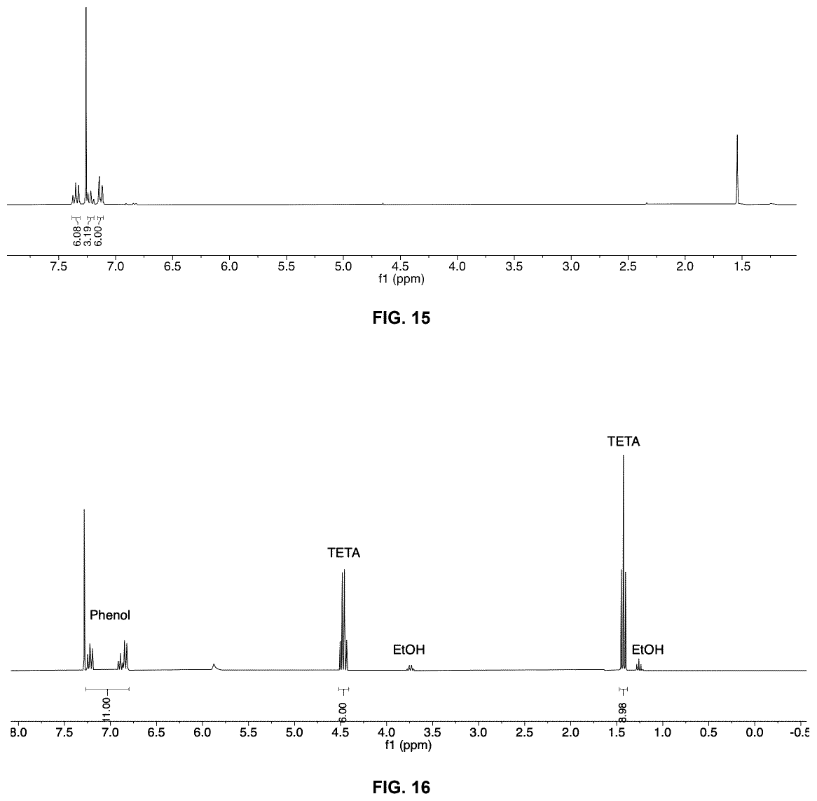

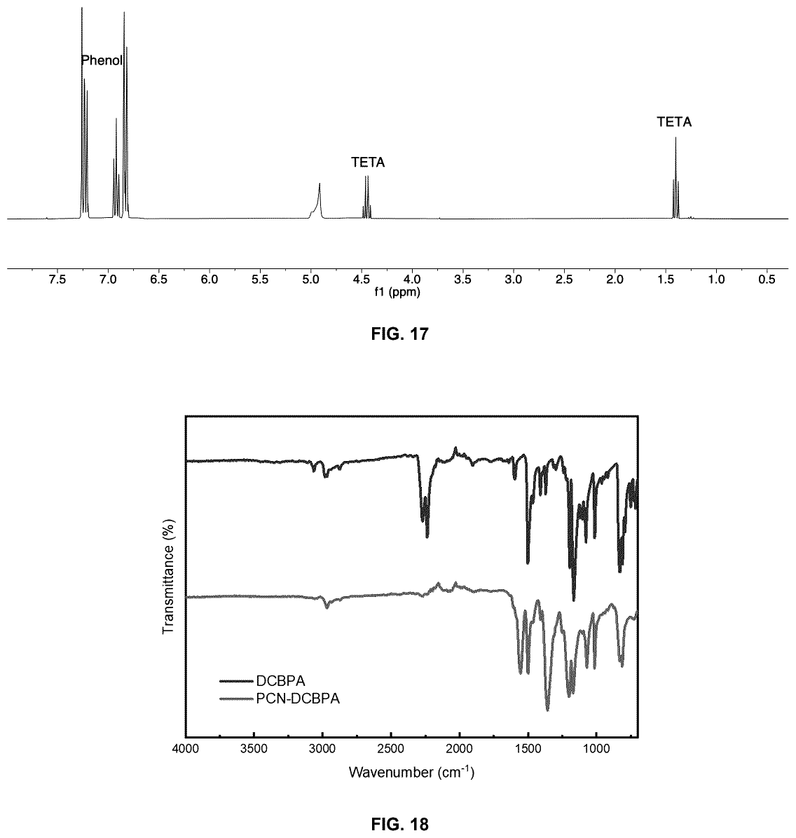

Next, the present inventors explored the materials' recyclability and found these PCNs can be efficiently converted back to monomers when refluxing in ethanol (FIG. 4a). For example, PCN-A6 could be slowly degraded in refluxing ethanol over two days. When 5 wt % of potassium carbonate was added as a base to deprotonate ethanol, the process was expedited, with PCNs degraded in ethanol within 16 hours with almost quantitative conversion into monomers. Both monomers, diols and TETA, can be easily recovered in ˜90% isolated yield from the mixture after removal of ethanol. Long-chain diols (e.g., 1,12-dodecanediol) precipitated out from the mixture upon addition of hexanes, providing clean diols as a solid. TETA can be recovered from hexanes solution in high purity (FIG. 9-11) and directly reused. Short-chain diols (e.g., 1,4-butane diol) can be separated from TETA through liquid-liquid extraction with hexanes to afford highly concentrated crude products that can be further purified via distillation.

To demonstrate the possibility of selectively recycling PCNs in a mixed plastic waste stream, the present inventors used a sample containing an equal amount of the mixed plastics, PCN-A6, PP (polypropylene), HDPE (high density polyethylene) and PS (polystyrene). After refluxing the plastic mixture with potassium carbonate (5 wt %) in ethanol, PCN-A6 completely depolymerized into TETA and 1,6-hexane diol, which can be separated from other plastics through filtration and extraction (FIG. 4b). Highly pure TETA (FIG. 4c) was recovered through simple evaporation and extraction. Recycled PCNs (PCN-Ax-Re) (x=4, 6 or 12) prepared using recovered TETA exhibit identical FT-IR absorption as the virgin PCNs (FIG. 9-11). The mechanical properties and T_{g} of the recycled PCNs are also highly comparable to those of the original ones (FIG. 4d, FIG. 4e and Table 4). A similar recycling method was also used to depolymerize the traditional aryl polycyanurate wastes such as PCN-DCBPA to form high purity TETA (FIG. 3a). These results clearly show our strategy of utilizing S_{N}Ar reaction is generally applicable to a broad range of PCNs, providing user-friendly closed-loop recyclability of this important class of thermosets.

### Example 4: Materials and Methods

General procedure for PCN film synthesis: TETA (1.0 eq.), diol (1.5 eq.), and TBD catalyst (0.06 eq.) were stirred in anisole at 100° C. for 15-30 minutes. The resulting homogeneous solution was then poured into a glass petri dish. The solvent was allowed to slowly evaporate in an oven at 120° C. for 14 hours, yielding a transparent defect-free PCN film. The film was further cured with a heat press machine at 130° C. under ambient pressure for 4 hours.

Gel fraction and chemical resistant tests: A PCN film was cut into small rectangular pieces and submerged in different organic solvents or solutions. The mixture was kept at room temperature for 48 hours without disturbance. The solution was then decanted. For gel fraction test, the residue was washed with excessive solvent for five times. For the chemical resistant test, the residue was washed with water and acetone for three times. The remaining solid was dried in an oven at 120° C. for 4 hours and weighed. The weight difference of the sample before and after the treatment was calculated.

Degradation of PCN: A piece of PCN film, potassium carbonate (5 wt % of the PCN film), and the internal standard (1,3,5-trimethoxybenzene) were stirred in ethanol (˜400 wt % of the PCN film) at 90° C. for 16 hours. After the mixture was cooled to room temperature, an aliquot of the mixture was dried under high vacuum for 10 minutes. The degradation progress was monitored by ¹H-NMR spectroscopy. The degradation was clean and completed after 16 hours. The amount and ratio of the two monomers were also determined by comparing the proton resonance signals in the ¹H-NMR spectrum of the degradation residue.

General procedure for chemical recycling of TETA: A piece of PCN film and potassium carbonate (˜5 wt % of PCN film) were stirred in ethanol (˜400 wt % of PCN film) at 90° C. for 16 hours. After the mixture was cooled to room temperature, the solid was filtered and washed with additional ethanol. Most of ethanol was removed via rotary evaporation, but not to dryness to prevent repolymerization. High vacuum was then applied at room temperature to remove the ethanol residue. Hexanes (˜200 wt % of the original PCN film) was added. The diols have poor solubility in hexanes and thus can be separated from the solution. The mixture was sonicated for 5 minutes. For PCN-A12 recycling, the resulting suspension was filtrated, and the solid residue was washed with additional hexanes and water to give the recycled DO-12. The filtrate was transferred to a vial and all the volatiles were removed by rotary evaporation to give TETA as an off-white crystal. For PCN-A4 and PCN-A6 recycling, the liquid mixture was washed two more times with fresh hexanes to completely extract TETA. The combined hexanes filtrate was washed with brine, dried over anhydrous Na₂SO₄, and concentrated to give TETA as an off-white crystal.

Chemical recycling of TETA from plastic mixtures: High density polyethylene (410 mg) from a flask cap, polypropylene (410 mg) from a tape case, polystyrene (581 mg) from a centrifuge tube, PCN-A6 (539 mg), and potassium carbonate (27 mg) were weighted to a 40 mL vial. Ethanol (20 mL) was added, and the mixture was stirred at 90° C. for 16 hours. After cooling to room temperature, the mixture was filtered. The remaining solid was washed with an additional 5 mL of ethanol. The filtrate was treated as described above for PCN recycling. TETA was recovered as an off-white crystal (387 mg, 85%).

### REFERENCES

1 Coates, G. W. & Getzler, Y. D. Y. L. Chemical recycling to monomer for an ideal, circular polymer economy. Nat. Rev. Mater. 5, 501-516, doi:10.1038/s41578-020-0190-4 (2020).
2 Vollmer, I. et al. Beyond Mechanical Recycling: Giving New Life to Plastic Waste. Angewandte Chemie International Edition 59, 15402-15423, doi:10.1002/anie.201915651 (2020).
3 Korley, L. T. J., Epps, T. H., 3rd, Helms, B. A. & Ryan, A. J. Toward polymer upcycling-adding value and tackling circularity. Science 373, 66-69, doi:10.1126/science.abg4503 (2021).
4 Garcia, J. M. et al. Recyclable, strong thermosets and organogels via paraformaldehyde condensation with diamines. Science 344, 732-735, doi:10.1126/science.1251484 (2014).
5 Zhu, J.-B., Watson, E. M., Tang, J. & Chen, E. Y.-X. A synthetic polymer system with repeatable chemical recyclability. Science 360, 398-403, doi:10.1126/science.aar5498 (2018).
6 Christensen, P. R., Scheuermann, A. M., Loeffler, K. E. & Helms, B. A. Closed-loop recycling of plastics enabled by dynamic covalent diketoenamine bonds. Nat. Chem. 11, 442-448, doi:10.1038/s41557-019-0249-2 (2019).
7 Shieh, P. et al. Cleavable comonomers enable degradable, recyclable thermoset plastics. Nature 583, 542-547, doi:10.1038/s41586-020-2495-2 (2020).
8 Abel, B. A., Snyder, R. L. & Coates, G. W. Chemically recyclable thermoplastics from reversible-deactivation polymerization of cyclic acetals. Science 373, 783-789, doi:10.1126/science.abh0626 (2021).
9 Haussler, M., Eck, M., Rothauer, D. & Mecking, S. Closed-loop recycling of polyethylene-like materials. Nature 590, 423-427, doi:10.1038/s41586-020-03149-9 (2021).
10 Mohadjer Beromi, M. et al. Iron-catalysed synthesis and chemical recycling of telechelic 1,3-enchained oligocyclobutanes. Nat. Chem. 13, 156-162, doi:10.1038/s41557-020-00614-w (2021).
11 Sathe, D. et al. Olefin metathesis-based chemically recyclable polymers enabled by fused-ring monomers. Nat. Chem. 13, 743-750, doi:10.1038/s41557-021-00748-5 (2021).
12 Geyer, R., Jambeck, J. R. & Law, K. L. Production, use, and fate of all plastics ever made. Sci. Adv. 3, e1700782, doi:10.1126/sciadv.1700782 (2017).
13 MacLeod, M., Arp, H. P. H., Tekman, M. B. & Jahnke, A. The global threat from plastic pollution. Science 373, 61-65, doi:10.1126/science.abg5433 (2021).
14 Garcia, J. M. & Robertson, M. L. The future of plastics recycling. Science 358, 870, doi:10.1126/science.aaq0324 (2017).
15 Ragaert, K., Delva, L. & Van Geem, K. Mechanical and chemical recycling of solid plastic waste. Waste Manage. (Oxford) 69, 24-58, doi:10.1016/j.wasman.2017.07.044 (2017).
16 Eagan, J. M. et al. Combining polyethylene and polypropylene: Enhanced performance with PE/i PP multiblock polymers. Science 355, 814-816, doi:10.1126/science.aah5744 (2017).
17 Jin, Y., Yu, C., Denman, R. J. & Zhang, W. Recent advances in dynamic covalent chemistry. Chem. Soc. Rev. 42, 6634-6654, doi:10.1039/c3cs60044k (2013).
18 Kloxin, C. J. & Bowman, C. N. Covalent adaptable networks: smart, reconfigurable and responsive network systems. Chem. Soc. Rev. 42, 7161-7173, doi: 10.1039/c3cs60046g (2013).
19 Denissen, W., Winne, J. M. & Du Prez, F. E. Vitrimers: permanent organic networks with glass-like fluidity. Chem. Sci. 7, 30-38 (2016).
20 Fortman, D. J. et al. Approaches to Sustainable and Continually Recyclable Cross-Linked Polymers. ACS Sustainable Chem. Eng. 6, 11145-11159, doi:10.1021/acssuschemeng.8b02355 (2018).
21 Jin, Y., Lei, Z., Taynton, P., Huang, S. & Zhang, W. Malleable and Recyclable Thermosets: The Next Generation of Plastics. Matter 1, 1456-1493, (2019).
22 Taynton, P. et al. Heat- or Water-Driven Malleability in a Highly Recyclable Covalent Network Polymer. Adv. Mater. 26, 3938-3942, (2014).
23 Montarnal, D., Capelot, M., Tournilhac, F. & Leibler, L. Silica-Like Malleable Materials from Permanent Organic Networks. Science 334, 965-968, doi:10.1126/science.1212648 (2011).
24 Röttger, M. et al. High-performance vitrimers from commodity thermoplastics through dioxaborolane metathesis. Science 356, 62-65, doi:10.1126/science.aah5281 (2017).
25 Cash, J. J., Kubo, T., Bapat, A. P. & Sumerlin, B. S. Room-Temperature Self-Healing Polymers Based on Dynamic-Covalent Boronic Esters. Macromolecules 48, 2098-2106, doi:10.1021/acs.macromol.5b00210 (2015).
26 Denissen, W. et al. Vinylogous Urethane Vitrimers. Adv. Funct. Mater. 25, 2451-2457, (2015).
27 Fortman, D. J., Brutman, J. P., Cramer, C. J., Hillmyer, M. A. & Dichtel, W. R. Mechanically Activated, Catalyst-Free Polyhydroxyurethane Vitrimers. J Am. Chem. Soc. 137, 14019-14022, doi:10.1021/jacs.5b08084 (2015).
28 Nishimura, Y., Chung, J., Muradyan, H. & Guan, Z. Silyl Ether as a Robust and Thermally Stable Dynamic Covalent Motif for Malleable Polymer Design. J. Am. Chem. Soc. 139, 14881-14884, doi:10.1021/jacs.7b08826 (2017).
29 Yue, L., Amirkhosravi, M., Gong, X., Gray, T. G. & Manas-Zloczower, I. Recycling Epoxy by Vitrimerization: Influence of an Initial Thermoset Chemical Structure. ACS Sustainable Chem. Eng. 8, 12706-12712, doi:10.1021/acssuschemeng.0c04815 (2020).
30 Yang, H., Jin, Y., Du, Y. & Zhang, W. Application of alkyne metathesis in polymer synthesis. J. Mater. Chem. A 2, 5986-5993, doi:10.1039/C3TA14227B (2014).
31 Gross, D. E., Discekici, E. & Moore, J. S. Macrocyclic depolymerization of arylene-ethynylene copolymers: a dynamic combinatorial method. Chem. Commun. 48, 4426-4428, doi:10.1039/c2cc30701d (2012).
32 Ramdani, N., Zaimeche, H. & Derradji, M. Biobased thermally-stable aromatic cyanate ester thermosets: A review. React. Funct. Polym. 168, 105037, (2021).
33 Kandelbauer, A. in Handbook of Thermoset Plastics (Third Edition) (eds Hanna Dodiuk & Sidney H. Goodman) 425-457 (William Andrew Publishing, 2014).
34 Hamerton, I. Chemistry and Technology of Cyanate Ester Resins. (Springer Netherlands, 1994).
35 Bauer, M. & Composite, F.-E. f P. u. Thermosets 2013 —From Monomers to Components: Proceedings of the 3rd International Conference on Thermosets, 18-20 Sep. 2013, Berlin, Germany. (Fraunhofer Research Institution for Polymeric Materials and Composites PYCO, 2013).
36 Söthje, D., Bauer, M. & Dreyer, C. Process for reutilization of recycled polycyanurate-containing materials in new polymeric materials. International patent (2014).
37 Dreyer, C. & Dominik, S. Progress in recycling of composites with polycyanurate matrix. Advances in Chemical Engineering and Science 2014 (2014).
38 Fainleib, A. M., Hourston, D. J., Grigoryeva, O. P., Shantalii, T. A. & Sergeeva, L. M. Structure development in aromatic polycyanurate networks modified with hydroxyl-terminated polyethers. Polymer 42, 8361-8372, (2001).
39 Rohrbach, S. et al. Concerted Nucleophilic Aromatic Substitution Reactions. Angew. Chem., Int. Ed 58, 16368-16388, doi:10.1002/anie.201902216 (2019).
40 Ong, W. J. & Swager, T. M. Dynamic self-correcting nucleophilic aromatic substitution. Nat. Chem. 10, 1023-1030, doi:10.1038/s41557-018-0122-8 (2018).
41 Jin, Y. & Zhang, W. S_{N}Ar stands corrected. Nat. Chem. 10, 996-998, doi:10.1038/s41557-018-0138-0 (2018).
42 Armarego, W. L. F. Purification of Laboratory Chemicals. (Elsevier Science, 2017).
43 Federico, S. et al. 5, 7-Disubstituted-[1, 2, 4]triazolo[1,5-a][1, 3, 5]triazines as pharmacological tools to explore the antagonist selectivity profiles toward adenosine receptors. Eur. J. Med. Chem. 108, 529-541 (2016).
44 Thorat, M. T., Mane, R. B., Jagdale, M. H., Salunkhe, M. M. & Wadgaonkar, P. P. Synthesis of triaryl cyanurates using polymer-supported reagents. Eur. Polym. J. 24, 399-401, (1988).
45 Liu, J. et al. A high-performance polycyanurate network derived from 4,4′-biscyanato-2,2′-trifluoromethylbiphenyl. Polym. Chem. 11, 784-788, doi:10.1039/C9PY01610D (2020).
46 Guenthner, A. et al. The Influence of Monomer Chemical Structure on Late-Stage Cure Kinetics of Dicyanate Ester Resins. International SAMPE Technical Conference (2011).

1. A composition comprising an alkyl- and/or aryl-linked crosslinked polymeric compound according to Formula (I) comprising: 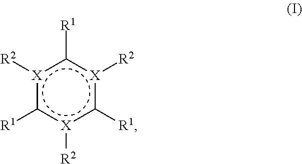
wherein
X is independently N, or C, and further when X are all N, then R² is absent, and when X is all C then R² is present;
R¹ is independently CH, halogen, alkyl, or diol selected from alkyl diol or aryl diol, and wherein at least two of R¹ are independently alkyl diol, or aryl diol;
R² is independently H, CH, or an electron withdrawing group, and wherein at least two of R² are independently an electron withdrawing group;
wherein any of R¹ and R² optionally form together one or more an aromatic rings, or one or more heterocyclic rings, and wherein the one or more rings are optionally substituted with at least one electron withdrawing group, and wherein the one or more rings of the compound further form an electron deficient ringed core; and
wherein said dashed lines represent possible double bond positions according to the configuration of X being N or C, wherein the double bond positions form an aromatic ring.

2. The compound of claim 1, wherein R¹ is selected from: polyol, polythiol, bisphenol A, polyamine, 1,4-butandiol, 1,6-hexandiol, and 1,12-dodecanediol, or a combination of the same.

3. The compound of claim 1, R¹ is selected from: 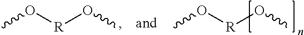
wherein
R is C₄₋₁₂ linear alkyl, an aromatic diol, polyol, polythiol or polyamine; and
n is greater than one.

4. The compound of claim 1, wherein the electron withdrawing group is selected from:
NO₂, CN, CHO, halogen, CO₂R³, CONR³, CH═NR³, (C═S)OR³, (C═O)SR³, CS₂R³, SO₂R³, SO₂NR³, SO₃R³, P(O)(OR³)₂, P(O)(R³)₂, or B(OR³)₃, wherein R³ is an alkyl, an aryl or H.

5. The compound of claim 1, where the electron withdrawing group is selected from: CN, CHO, or halogen.

6. The compound of claim 1, wherein the compound is selected from:

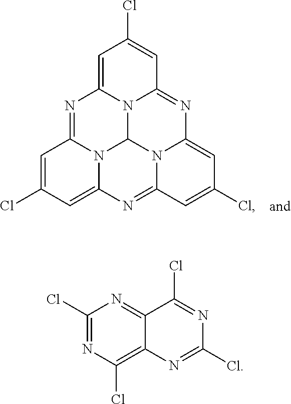 [SMILES: ClC1=CC2=NC3=CC(Cl)=CC4=NC5=CC(Cl)=CC6=NC(=C1)N2C(N34)N56 ; Clc1nc(Cl)c2nc(Cl)nc(Cl)c2n1]

7. A compound comprising an alkyl-linked polycyanurate network (PCN) formed by a plurality of alkyl-linked polycyanurate compounds according to Formula (II): 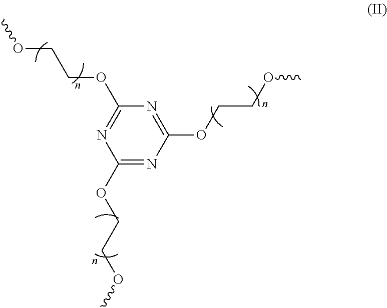
wherein n is greater than 1.

8. The compound of claim 7, wherein n is between 2-6.

9. An alkyl-linked polycyanurate network (PCN) formed by the monomer unit compounds of claim 7.

10. A compound comprising an alkyl-linked polyarylether network (PAE) formed by a plurality of alkyl-linked polyether compounds according to Formula (III): 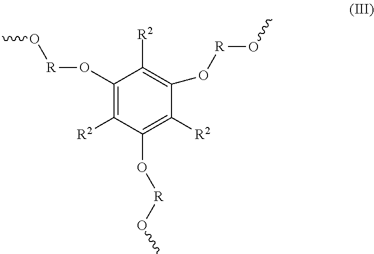
wherein
R is independently alkyl or aryl; and
R² is independently an electron withdrawing group.

11. The compound of claim 10, wherein R is a C₄₋₁₂ linear alkyl.

12. The compound of claim 10, wherein the electron withdrawing group is selected from: NO₂, CN, CHO, halogen, CO₂R³, CONR³, CH═NR³, (C═S)OR³, (C═O)SR³, CS₂R³, SO₂R³, SO₂NR³, SO₃R³, P(O)(OR³)₂, P(O)(R³)₂, or B(OR³)₃type wherein R³ is an alkyl, an aryl or a hydrogen atom.

13. The compound of claim 10, where the electron withdrawing group is selected from: CN, CHO, or halogen.

14. An alkyl-linked polyarylether network (PAE) formed by the monomer unit compounds of claim 10.

15-18. (canceled)

19. An alkyl- or aryl-linked polycyanurate compound according to Formula (IA) comprising: 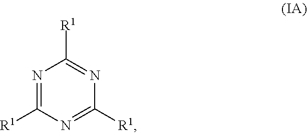
wherein R¹ is an alkyl or aryl diol.

20. The compound of claim 19, wherein R¹ comprises: 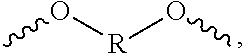
wherein R is linear alkyl.

21. The compound of claim 20, wherein R is a C₄₋₁₂ linear alkyl.

22. The compound of claim 19, wherein R¹ comprises bisphenol A

23. The compound of claim 19, wherein said R¹ is selected from: 1,4-butandiol, 1,6-hexandiol and 1,12-dodecanediol, and bisphenol A.

24-32. (canceled)
